(19) United States
(12) Patent Application Publication
(10) Pub. No.: US 2025/0066359 A1
(43) Pub. Date: Feb. 27, 2025

(54) SOMATOSTATIN RECEPTOR SUBTYPE 5 ANTAGONISTS, AND PHARMACEUTICAL COMPOSITION AND USE THEREOF

(71) Applicant: SHANGHAI INSTITUTE OF MATERIA MEDICA, CHINESE ACADEMY OF SCIENCES, Shanghai (CN)

(72) Inventors: Jianhua SHEN, Shanghai (CN); Xin XIE, Shanghai (CN); Yanglong ZHANG, Shanghai (CN); Tingting ZHAO, Shanghai (CN); Fanghui HAN, Shanghai (CN); Ying YUN, Shanghai (CN)

(21) Appl. No.: 18/724,366

(22) PCT Filed: Dec. 27, 2022
(86) PCT No.: PCT/CN2022/142195

§ 371 Date: Aug. 26, 2024

(30) Foreign Application Priority Data
Dec. 27, 2021 (CN) 202111613181.1
Dec. 13, 2022 (CN) 202211603287.8

(51) Int. Cl. C07D 471/10; A61K 31/397; A61K 31/407; A61K 31/437; A61K 31/438; A61K 31/444; A61K 31/4523; A61K 31/495; A61K 31/496; A61K 31/5386; A61K 45/06; A61P 1/16; A61P 3/10; C07D 205/12; C07D 211/96; C07D 211/98; C07D 221/20; C07D 295/26; C07D 401/04; C07D 471/04; C07D 487/10; C07D 498/10

(52) U.S. Cl. CPC C07D 471/10; A61K 31/397; A61K 31/407; A61K 31/437; A61K 31/438; A61K 31/444; A61K 31/4523; A61K 31/495; A61K 31/496; A61K 31/5386; A61K 45/06; A61P 1/16; A61P 3/10; C07D 205/12; C07D 211/96; C07D 211/98; C07D 221/20; C07D 295/26; C07D 401/04; C07D 471/04; C07D 487/10; C07D 498/10

(57) ABSTRACT

Provided are a class of compounds which have a structure as shown in the following general formula (I) and are used as somatostatin receptor subtype 5 (SSTR5) antagonists, and a pharmaceutical composition and the use thereof. The compounds have good SSTR5 antagonistic activities, and can be used for preparing drugs for treating related diseases mediated by SSTR5.

### TECHNICAL FIELD

The present invention belongs to the field of pharmacy, and specifically relates to a kind of somatostatin receptor subtype 5 antagonists, a pharmaceutical composition, and pharmaceutical use thereof.

### BACKGROUND

Somatostatin Receptor subtype 5 (SSTR5), an inhibitory G protein-coupled receptor, is distributed mainly in the pituitary gland, gastrointestinal tract and pancreatic islets in rodents, and highly distributed in the gastrointestinal tract in humans (Regulatory peptides, 2000, 90(1-3): 1-18). Its endogenous ligand is somatostatin (SST), which is mainly divided into SST-14 and SST-28. SST can activate SSTR5 by binding to SSTR5, mediating the inhibitory effect of hormone secretion. Among them, SSTR5 activation in gastrointestinal tract can inhibit the secretion of gastrointestinal hormones such as GLP-1, GLP-2, GIP, PYY, CCK, etc.; SSTR5 activation in pancreatic islet tissue can inhibit the secretion of insulin. (Frontiers in Neuroendocrinology, 34 (2013) 228-252; Am J Physiol Gastrointest Liver Physiol 279: G983-G989, 2000); Pharmacological studies have shown that SSTR5 antagonists can antagonize the SSTR5 activation effect mediated by the binding of SSTR5 to endogenous ligands, thereby promoting the secretion of gastrointestinal hormones such as GLP-1, GLP-2, GIP, PYY, CCK, etc. (Diabetologia, 55 (2012) 3094-3103), as well as promoting insulin secretion and having a positive effect on gallbladder motility. At the same time, the blood glucose processing ability and insulin resistance effect in the SSTR5 gene knockout mouse model were significantly improved, compared with that in wild-type mice (Molecular Endocrinology 17(1):93-106). GLP-1 has multiple physiological functions, such as promoting blood glucose-dependent insulin secretion and inhibiting glucagon secretion, promoting satiety, slowing gastric emptying, and playing a protective role in liver, kidney, and myocardium. Currently, GLP-1-based drugs have been successfully used in the fields of type II diabetes and obesity; at the same time, they have shown positive therapeutic effects in clinical trials of non-alcoholic fatty liver disease, Alzheimer's disease, and Parkinson's disease. GLP-2 can promote small intestinal growth and nutrient absorption, which is essential for maintaining intestinal homeostasis. GLP-2 analogs have been approved for short bowel syndrome and have shown positive therapeutic effects in animal models of inflammatory bowel disease. GIP mainly acts on the pancreatic islets to exert blood glucose-dependent blood glucose homeostasis regulation function and has a synergistic effect with GLP-1. PYY can slow gastric emptying and promote satiety, and is used to treat obesity. CCK can promote gallbladder contraction and promote bile outflow from gallbladder (Curr Med Chem. 2019; 26(19): 3407-3423), wherein the gallbladder emptying function is related to a variety of gallbladder diseases (GASTROENTEROLOGY 1996; 111:765-771; Laboratory Investigation (2015) 95, 124-131), such as gallstones, cholestasis and primary sclerosing cholangitis. Therefore, antagonizing SSTR5 is a potential therapy for gallstones, cholestasis and primary sclerosing cholangitis.

Further studies have shown that SSTR5 antagonists have significant synergistic effects with receptor agonists that promote gastrointestinal hormone secretion (such as TGR5, GPR40, GPR119, GPR41, GPR43 agonists, etc.) and DPP4 inhibitors that inhibit degradation. The combined use of the three can significantly increase the level of gastrointestinal hormones (Diabetes 2018 February; 67(2): 309-320). Therefore, STTR5 antagonists can be used in combination with TGR5 agonists, GPR40 full agonists, GPR119 agonists, GPR41 agonists, GPR43 agonists and DPP4 inhibitors.

In summary, it is necessary to develop a kind of SSTR5 antagonists with a novel structure, which are to be used in the treatment of chronic metabolic diseases such as type II diabetes, non-alcoholic fatty liver disease (NAFLD), non-alcoholic steatohepatitis (NASH), inflammatory bowel disease, obesity, gallstones, cholangitis, etc. At the same time, it has good clinical application prospects when being used in combination with TGR5 agonists, GPR40 modulators, GPR119 agonists, GPR41 agonists, GPR43 agonists and DPP4 inhibitors for diseases related to GLP-1 and GIP, such as type II diabetes, obesity, non-alcoholic fatty liver disease, non-alcoholic fatty liver fibrosis, Parkinson's disease and Alzheimer's disease, etc.

### SUMMARY OF THE INVENTION

One technical purpose of the present invention is to provide a kind of compounds having somatostatin receptor 5 antagonism.

Another technical purpose of the present invention is to provide a pharmaceutical composition comprising the compound.

Another technical purpose of the present invention is to provide use of the compound or the pharmaceutical composition in the preparation of a somatostatin receptor 5 antagonist.

In the first aspect, the present disclosure provides a compound according to the following general formula I, or a solvate, hydrate, deuterated compound, stereoisomer, tautomer, or pharmaceutically acceptable salt thereof:

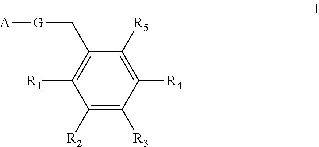

Wherein, R₁, R₂, R₃, R₄, and R₅ are each independently selected from the group consisting of hydrogen, hydroxyl, C₁-C₆ alkyl, C₁-C₆ alkoxy, C₃-C₆ cycloalkyl, halogen, C₁-C₆ haloalkyl, —OH, —NH₂, —N(C₁-C₃ alkyl)(C₁-C₃ alkyl), —NH(C₁-C₃ alkyl), and substituted or unsubstituted C₆-C₁₄ aryl; wherein the substitution means that one or more hydrogen atoms on the aryl group is substituted by a group selected from the group consisting of halogen, C₁-C₃ haloalkoxy, C₁-C₃ alkyl, C₁-C₃ haloalkyl, C₁-C₃ haloalkoxy, C₃-C₆ cycloalkyl, —OH, —NH₂, —NH(C₁-C₃ alkyl), and —N(C₁-C₃ alkyl)(C₁-C₃ alkyl);
alternatively, any two adjacent substituents of R₁, R₂, R₃, R₄ and R₅ together with the benzene ring form a benzo 5-7 membered heterocyclic ring containing N, O or S, or a benzo 5-7 membered carbocyclic ring, wherein the heterocyclic ring or carbocyclic ring is unsubstituted or substituted by one or more substituents selected from the group consisting of halogen, C₁-C₆ alkyl, C₁-C₆ haloalkyl, C₁-C₆ alkoxy, C₁-C₆ haloalkoxy, C₃-C₆ cycloalkyl, —OH, —NH₂, and —N(C₁-C₆ alkyl)(C₁-C₆ alkyl);
A-G is represented by the following formula III or IV:

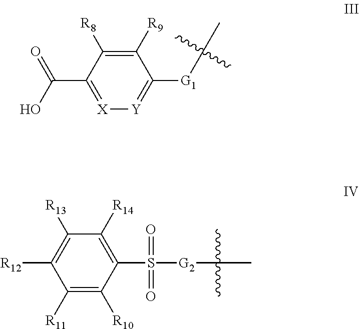

In formula III, R₈ and R₉ are each independently hydrogen, halogen, C₁-C₃ alkyl, C₁-C₃ alkoxy, C₁-C₃ haloalkoxy, —OH, —NH₂, —NH(C₁-C₃ alkyl), —N(C₁-C₃ alkyl)(C₁-C₃ alkyl); preferably, R₆, R₇, R₈ and R₉ are each independently selected from hydrogen, halogen and C₁-C₃ alkoxy;
X and Y are each independently CH or N;
G₁ is selected from the following structures, from the left side (A) to the right side (benzyl);

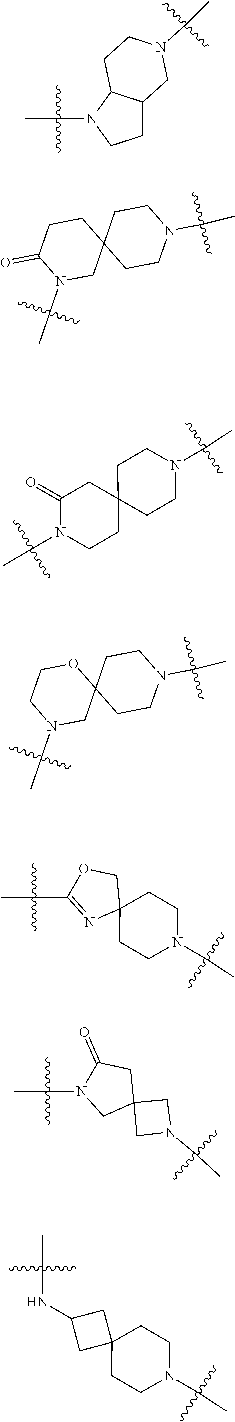

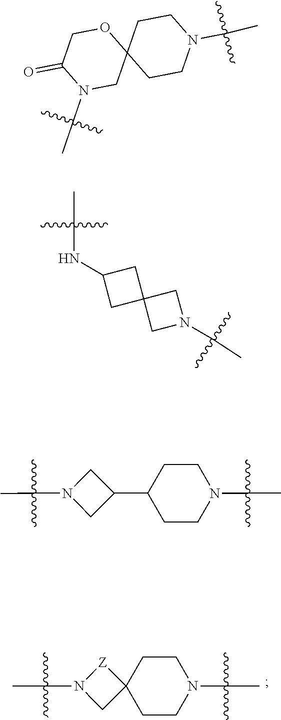

In formula IV, R₁₀, R₁₁, R₁₃ and R₁₄ are each independently hydrogen, halogen; R₁₂ is selected from carboxyl, C₁-C₃ haloalkoxy; G₂ is selected from the following structures, from the left side to the right side:

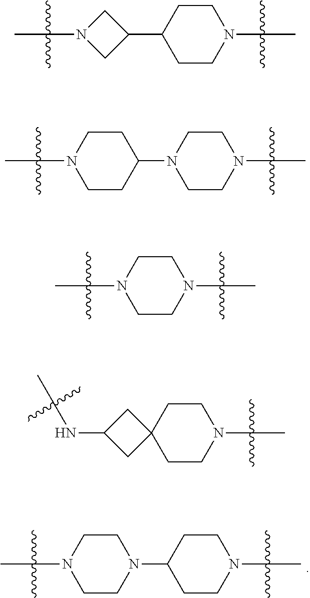 [SMILES: *N1CCC(C2CN(*)C2)CC1 ; *N1CCC(N2CCN(*)CC2)CC1 ; *N1CCN(*)CC1 ; *NC1CC2(CCN(*)CC2)C1 ; *N1CCC(N2CCN(*)CC2)CC1]

In a specific embodiment, the compound of formula I is represented by the following formula IIIa:

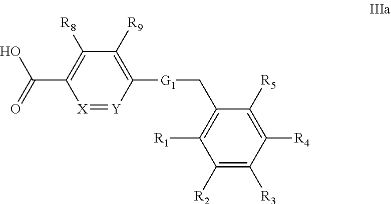

In the formula IIIa,
G₁ is selected from

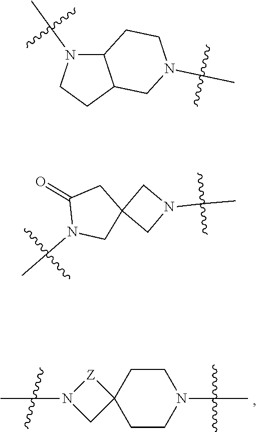

wherein Z is CH₂ or C═O; R₁ and R₅ are each independently selected from the group consisting of hydrogen, C₃-C₆ cycloalkyl, and substituted or unsubstituted phenyl; wherein the substitution means that the hydrogen on the phenyl is substituted by 1, 2 or 3 substituents selected from the group consisting of halogen, C₁-C₃ haloalkoxy, C₁-C₃ haloalkyl, C₁-C₃ alkoxy, and C₁-C₃ alkyl;
R₂ and R₄ are each independently selected from the group consisting of hydrogen, Cr C₃ alkoxy, halogen, and C₁-C₃ haloalkyl;
R₃ is selected from hydrogen, hydroxyl, halogen, C₁-C₃ alkyl, C₁-C₃ haloalkyl, —N(C₁-C₃ alkyl)(C₁-C₃ alkyl), and substituted or unsubstituted phenyl; wherein the substitution means that the phenyl is substituted by a group selected from the group consisting of halogen, C₁-C₃ haloalkoxy, C₁-C₃ haloalkyl, C₁-C₃ alkoxy, and C₁-C₃ alkyl;
R₈ and R₉ are each independently hydrogen, halogen, C₁-C₃ alkyl, C₁-C₃ alkoxy, or C₁-C₃ haloalkoxy,
X and Y are each independently CH or N.

In a specific embodiment, in the general formula IIIa,
G₁ is selected from

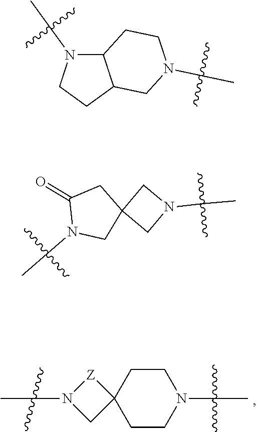

wherein Z is CH₂ or C═O;
R₁ and R₅ are each independently selected from hydrogen, C₃-C₆ cycloalkyl, and phenyl unsubstituted or substituted by 1, 2 or 3 halogens;
R₂ and R₄ are each independently selected from hydrogen, C₁-C₃ alkoxy, and C₁-C₃ haloalkyl;
R₃ is selected from hydrogen, hydroxyl, halogen, C₁-C₃ alkyl, —N(C₁-C₃ alkyl)(C₁-C₃ alkyl), and phenyl unsubstituted or substituted by halogen;
R₈ and R₉ are each independently hydrogen, halogen, or C₁-C₃ alkoxy,
X and Y are each independently CH or N.

In a specific embodiment, in the general formula IIIa,
G₁ is selected from

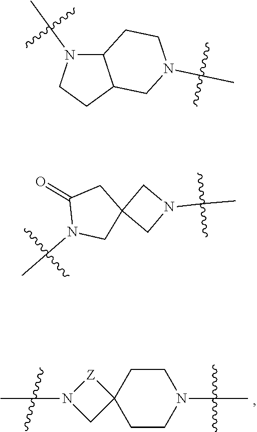

wherein Z is CH₂ or C═O;
R₁ and R₅ are selected from hydrogen, cyclopropyl, and phenyl substituted by 1 to 3 fluorines;
R₂ and R₄ are selected from hydrogen, ethoxy, and trifluoromethyl;
R₃ is selected from hydrogen, hydroxyl, fluorine, methyl, diethylamino, and p-fluorophenyl;
R₈ to R₉ are each independently selected from hydrogen, fluorine, and methoxy,
X and Y are each independently CH or N.

In a specific embodiment, in the general formula IIIa,
G₁ is selected from

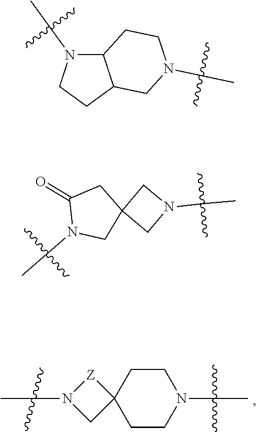

wherein Z is CH₂ or C═O;
R₁ and R₅ are hydrogen; R₂ and R₄ are ethoxy; R₃ is p-fluorophenyl; R₈ and R₉ are each independently selected from hydrogen, fluorine, and methoxy, X and Y are each independently CH or N.

In a specific embodiment, in the general formula IIIa,
one of R₁ and R₅ is hydrogen, and the other is cyclopropyl; one of R₂ and R₄ is ethoxy, and the other is hydrogen; R₃ is methyl; R₈ and R₉ are each independently selected from hydrogen, fluorine, and methoxy, and X and Y are each independently CH or N.

In another specific embodiment, the compound of formula I is represented by the following general formula IIIa1:

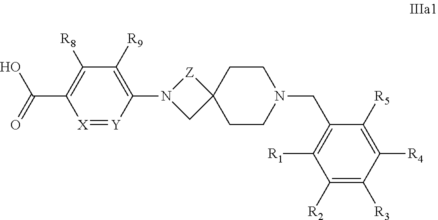

In the general formula IIIa1, each substituent is as defined above.

In a specific embodiment, R₁, R₂, R₄ and R₅ are each independently hydrogen, halogen, C₁-C₃ alkoxy, C₃-C₆ cycloalkyl, C₁-C₃ alkyl, or phenyl substituted by 1 to 3 halogens;
R₃ is hydrogen, C₁-C₃ alkyl, or phenyl substituted by 1 to 3 halogens;
R₈ and R₉ are each independently hydrogen, fluorine, C₁-C₃ alkoxy, or C₁-C₃ alkyl;
Z is CH₂ or C═O;
X and Y are each independently CH or N.

In another specific embodiment, the compound of the general formula I is represented by the following general formula IVa:

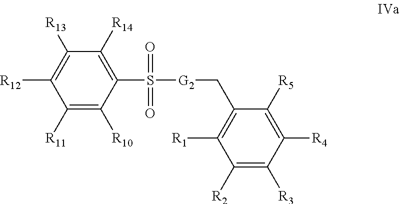

In the general formula IVa,
R₁ and R₅ are each independently selected from the group consisting of hydrogen, C₃-C₆ cycloalkyl, and substituted or unsubstituted phenyl; wherein the substitution means that the hydrogen on the phenyl is substituted by 1, 2 or 3 groups selected from the group consisting of halogen, C₁-C₃ haloalkoxy, C₁-C₃ haloalkyl, C₁-C₃ alkoxy, and C₁-C₃ alkyl;
R₂ and R₄ are each independently selected from the group consisting of hydrogen, C₁-C₃ alkoxy, halogen, and C₁-C₃ haloalkyl;
R₃ is selected from hydrogen, halogen, C₁-C₃ alkyl, C₁-C₃ haloalkyl, and substituted or unsubstituted phenyl; wherein the substitution means that the phenyl is substituted by a substituent selected from the group consisting of halogen, C₁-C₃ haloalkoxy, C₁-C₃ haloalkyl, C₁-C₃ alkoxy, and C₁-C₃ alkyl;
R₁₀, R₁₁, R₁₃ and R₁₄ are each independently hydrogen or halogen,
R₁₂ is selected from carboxyl, and C₁-C₃ haloalkoxy, G₂ is as defined above.

In a specific embodiment, in the general formula IVa,
R₁ and R₅ are hydrogen; R₂ and R₄ are ethoxy; R₃ is p-fluorophenyl; R₁₂ is selected from C₁-C₃ haloalkoxy and carboxyl; R₁₀, R₁₁, R₁₃ and R₁₄ are all hydrogen; G₂ is as defined above.

In a specific embodiment, in the general formula IVa,
R₁ and R₅ are hydrogen; R₂ and R₄ are ethoxy; R₃ is p-fluorophenyl; R₁₂ is selected from C₁-C₃ haloalkoxy and carboxyl; R₁₀, R₁₁, R₁₃ and R₁₄ are all hydrogen; G₂ is selected from

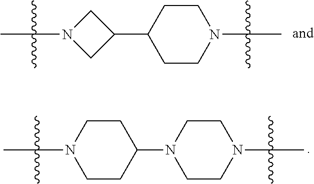 [SMILES: *N1CCC(C2CN(*)C2)CC1 ; *N1CCC(N2CCN(*)CC2)CC1]

In a specific embodiment, in the general formula IVa, R₁ and R₅ are hydrogen; R₂ and R₄ are ethoxy; R₃ is p-fluorophenyl; R₁₂ is trifluoromethoxy or carboxyl, R₁₀, R₁₁, R₁₃ and R₁₄ are all hydrogen; G₂ is as defined above.

In a specific embodiment, the compound of the general formula I is selected from one of the following compounds:

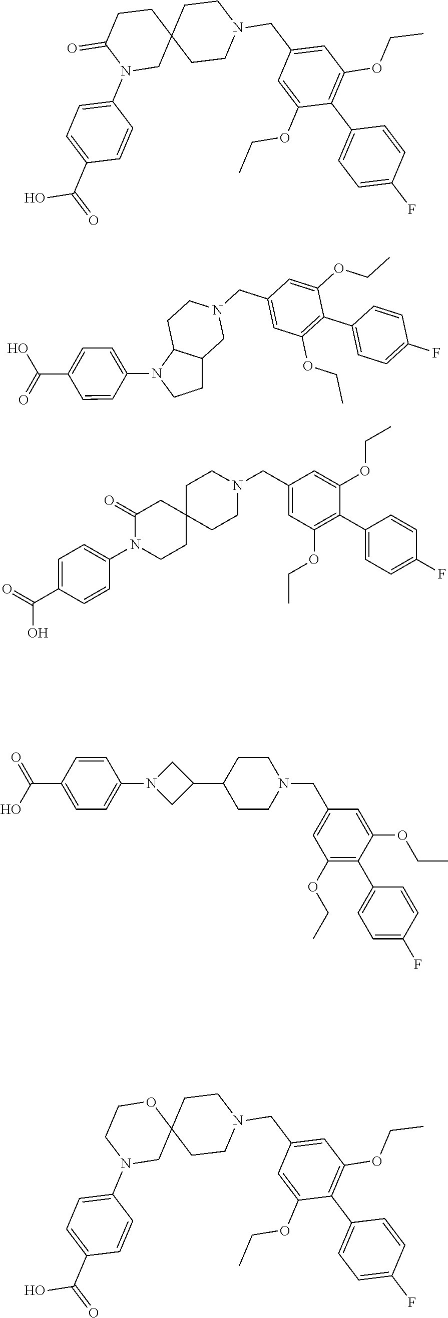

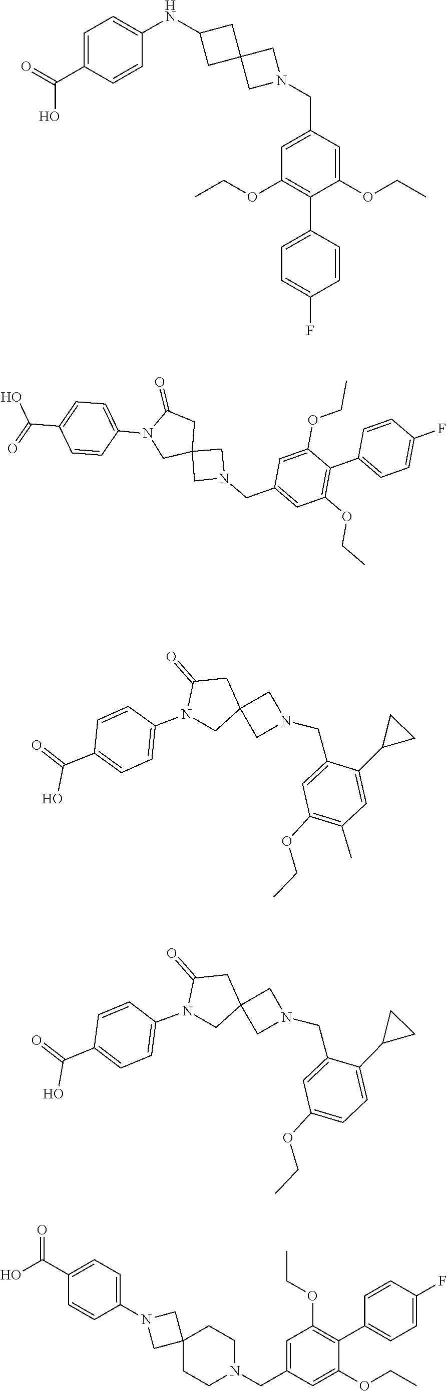

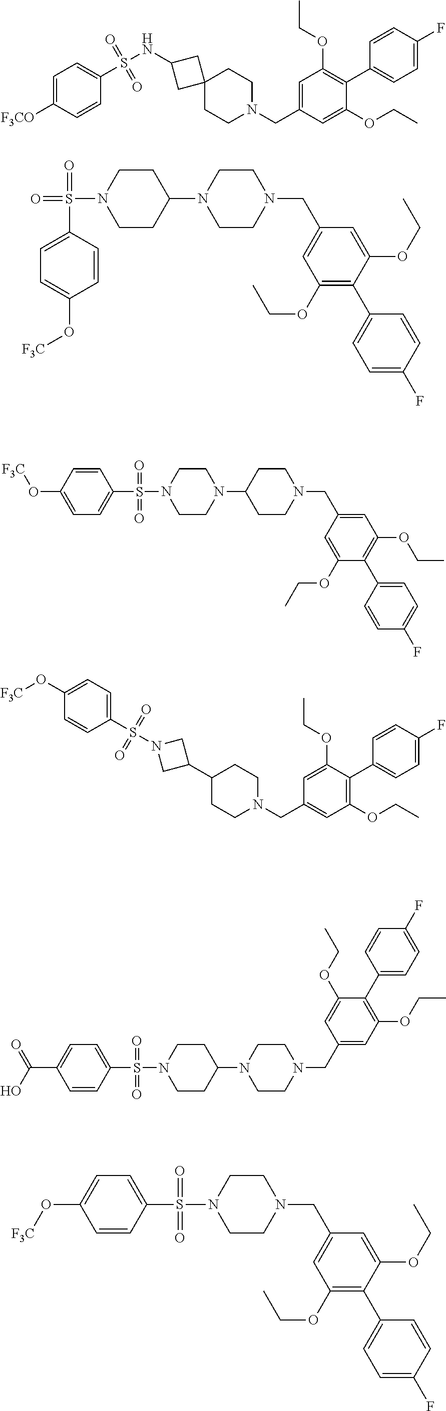

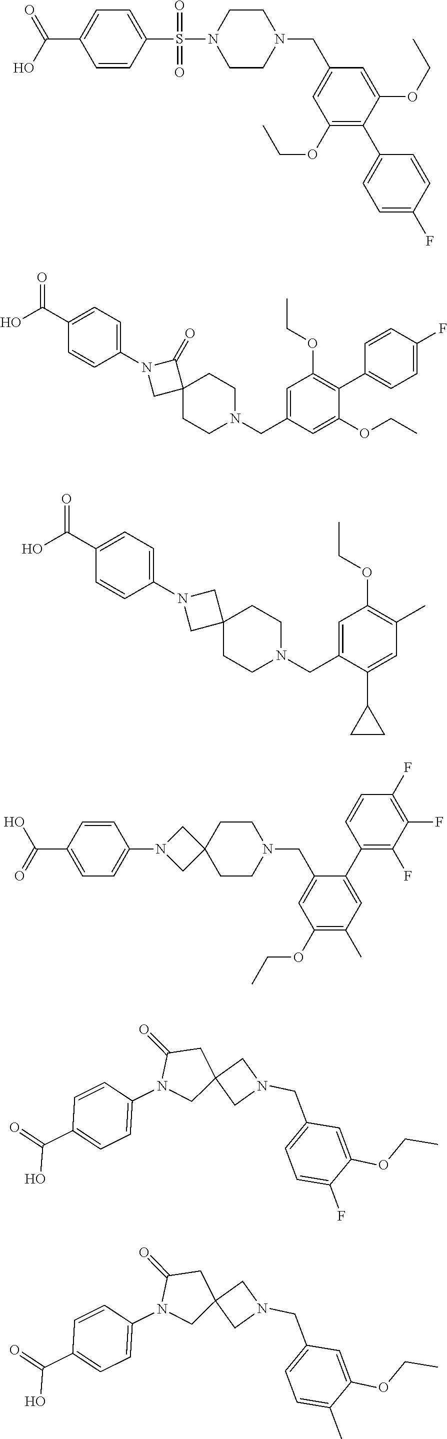

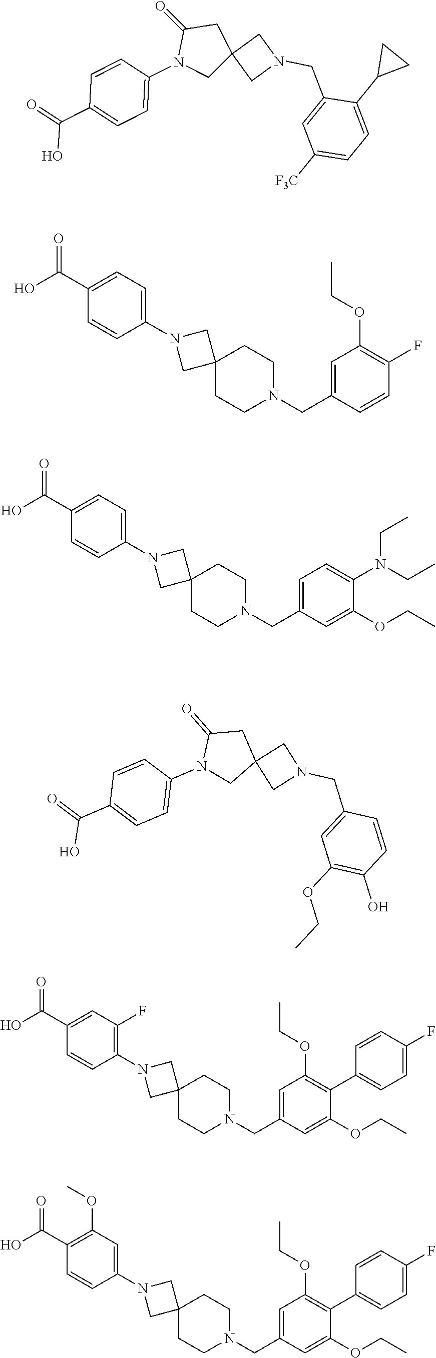

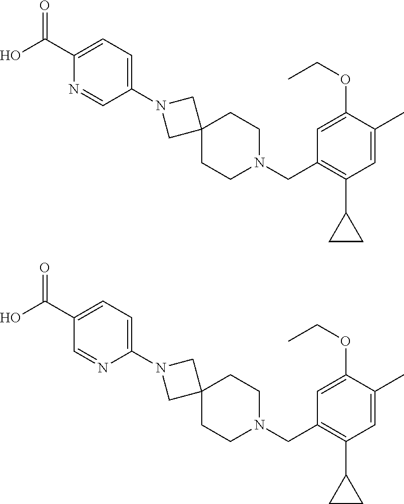

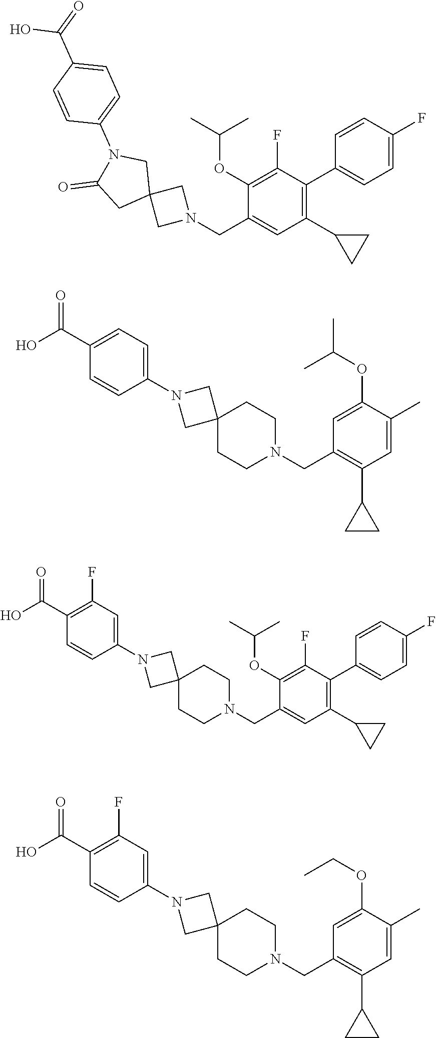

In the second aspect, the present disclosure provides a pharmaceutical composition comprising one or more therapeutically effective amounts of the compound of the general formula I or the solvate, hydrate, deuterated compound, stereoisomer, tautomer, pharmaceutically acceptable salt thereof as described above, and optionally a pharmaceutically acceptable excipient.

In a specific embodiment, the pharmaceutical composition further comprises a DPP4 inhibitor and one or more selected from a TGR5 agonist, a GPR40 agonist, a GPR119 agonist, a GPR41 agonist, and a GPR43 agonist.

In the third aspect, the present disclosure provides use of the compound of the general formula I, or the solvate, hydrate, deuterated compound, stereoisomer, tautomer, pharmaceutically acceptable salt thereof as described above, or the pharmaceutical composition as described above in the preparation of a drug for preventing or treating a SSTR5-mediated disease.

In a specific embodiment, the SSTR5-mediated disease includes type II diabetes, obesity, non-alcoholic fatty liver disease, gallbladder-related diseases, or inflammatory bowel disease.

In a specific embodiment, the non-alcoholic fatty liver disease is non-alcoholic steatohepatitis; and the gallbladder-related disease is selected from the group consisting of gallstones, primary sclerosing cholangitis, primary biliary cholangitis and cholestasis.

### Beneficial Effect

The present invention provides a kind of compounds with novel structures. Pharmacological studies have shown that the compounds of the present invention have good SSTR5 antagonistic activity and can be used to prepare drugs for treating related diseases mediated by SSTR5.

### BRIEF DESCRIPTION OF DRAWINGS

FIG. 1 shows the results of the blood glucose lowering test of the compound in Example 7 of the present disclosure, where * indicates P<0.05, and ** indicates P<0.01.

FIG. 2 shows the results of the gallbladder emptying test of compound 34 of the present disclosure.

### DETAILED EMBODIMENTS

### Definitions

The terms used in the present invention have the following definitions, unless otherwise specified:

In the present invention, the term “C₁-C₆” refers to having 1, 2, 3, 4, 5 or 6 carbon atoms, “C₁-C₄” refers to having 1, 2, 3, or 4 carbon atoms, and so on. “3-6 members” refers to having 3-6 ring atoms, and so on.

“Substitution” in the present invention means replacement by one or more groups (e.g., 2, 3, 4 or 5 groups). When multiple groups are selected from the same series of candidate substituents, they can be the same or different.

“Optionally” in the present invention means that the defined group can be selected from a series of candidate groups or not.

“Alkyl” in the present invention means saturated straight-chain and branched alkyl groups with a specific number of atoms, specifically, its examples include, but are not limited to, methyl, ethyl, n-propyl, isopropyl, n-butyl, isobutyl, sec-butyl, tert-butyl, pentyl, isopentyl, neopentyl, tert-pentyl, etc. The “C₁₋₃ alkyl” means a saturated straight or branched alkyl group with 1, 2 or 3 carbon atoms, and specifically its examples include, but are not limited to, methyl, ethyl, n-propyl, isopropyl, etc.

The “cycloalkyl” in the present invention means a non-aromatic, saturated, cyclic aliphatic hydrocarbon group with a specific number of carbon atoms as the ring atoms.

Representative examples of “C₃₋₆ cycloalkyl” include cyclopropyl, cyclobutyl, cyclopentyl, and cyclohexyl.

The “alkoxy” in the present invention means all straight-chain or branched alkoxy groups with a specific number of carbon atoms, and specifically its examples include, but are not limited to, methoxy, ethoxy, n-propoxy, isopropoxy, and n-butoxy.

The “halogen” refers to fluorine, chlorine, bromine, and iodine.

In the present invention, for the substitution on the alkyl or cycloalkyl group, if not specified to bind to a specific carbon atom, it means that it can bind to any carbon atom whose number of substituents has not yet reached saturation. When multiple substituents are selected from the same series, they can be the same or different.

In the present invention, the term “heterocyclic group” means a saturated cyclic group containing at least one ring heteroatom (such as N, O or S).

In the present invention, for the substitution on a benzene ring, aromatic heterocyclic ring or heterocyclic ring, if not specified to bind to a specific atom, it means that it can bind to any position not substituted by atoms other than hydrogen. When multiple substituents are selected from the same series, they can be the same or different.

“Pharmaceutically acceptable salt” means that the compound represented by formula (I) retains the desired biological activity and has minimal toxic side effects. The pharmaceutically acceptable salt can be obtained directly during the preparation and purification of the compound, or obtained indirectly by reacting the free acid or free base of the compound with another suitable base or acid.

The term “solvate” is used herein to describe a molecular complex comprising a compound of the present invention and a stoichiometric amount of one or more pharmaceutically acceptable solvent molecules (such as ethanol). When the solvent is water, the term “hydrate” is used.

### Pharmaceutical Composition

The compound of the present invention is usually administered in the form of a standard pharmaceutical composition when being used for treatment. It contains one or more therapeutically effective amounts of compounds represented by the general formula (I), and pharmaceutically acceptable excipients. The pharmaceutically acceptable excipients can be pharmaceutically acceptable carriers, excipients or sustained-release agents, etc.

The compounds and pharmaceutical compositions provided by the present invention can be in various forms, such as tablets, capsules, powders, syrups, solutions, suspensions and aerosols, etc., and can be present in suitable solid or liquid carriers or diluents. The pharmaceutical composition of the present invention can also be stored in a suitable sterilizing apparatus for injection or infusion. The pharmaceutical composition may also contain odorants, flavoring agents, etc.

In the present invention, the pharmaceutical composition contains a safe and effective amount (such as 0.1 to 99.9 parts by weight, preferably 1 to 90 parts by weight) of a compound represented by the general formula (I) or a pharmaceutically acceptable salt thereof; and a residual amount of pharmaceutically acceptable excipients, wherein the total weight of the composition is 100 parts by weight. Alternatively, the pharmaceutical composition of the present invention contains 0.1 to 99.9% by weight, preferably 1 to 90% by weight, of the compound represented by the general formula (I) or a pharmaceutically acceptable salt thereof; and a residual amount of pharmaceutically acceptable excipients, wherein the total weight of the composition is 100% by weight.

The preferred ratio of the compound represented by the general formula (I) to the pharmaceutically acceptable carrier, excipient or sustained-release agent is that the compound represented by the general formula (I) as an active ingredient accounts for more than 60% of the total weight, and the remaining part accounts for 0 to 40% of the total weight, and the amount of the remaining part is preferably 1 to 20%, and most preferably 1 to 10%.

The compound represented by the general formula (I) or the pharmaceutical composition containing the compound represented by the general formula (I) can be administered clinically to mammals, including humans and animals, and the route of administration may include oral administration, nasal inhalation, transdermal absorption, pulmonary administration or gastrointestinal tract, etc. The preferred route of administration is oral administration. It is preferable that a unit dosage form, with each dose containing 0.01 mg to 200 mg of active ingredient, preferably 0.5 mg to 100 mg of active ingredient, is taken once or in divided doses. Regardless of the route of administration, the optimal dose for an individual should be determined according to the specific treatment. In normal cases, it is started at a small dose and gradually increased until the most suitable dose is found.

The pharmaceutical composition of the present invention can be administered orally as well as intravenously, intramuscularly or subcutaneously, etc. Considering the ease of preparation and administration, the preferred pharmaceutical composition is a solid composition, especially tablets and solid-filled or liquid-filled capsules. Oral administration of the pharmaceutical composition is preferred.

The solid carriers include: starch, lactose, dicalcium phosphate, microcrystalline cellulose, sucrose and kaolin, etc., while the liquid carriers include: sterile water, polyethylene glycol, non-ionic surfactants and edible oils (such as corn oil, peanut oil and sesame oil), etc., as long as they are suitable for the characteristics of the active ingredient and the specific administration manners required. Adjuvants commonly used in the preparation of pharmaceutical compositions may also be advantageously included, such as flavoring agents, pigments, preservatives and antioxidants such as vitamin E, vitamin C, BHT and BHA.

Injectable preparations include, but are not limited to, sterile, injectable, aqueous, oily solutions, suspensions, emulsions, etc. These preparations can also be formulated with suitable parenteral diluents, dispersants, wetting agents, or suspending agents, etc. Such injectable preparations can be sterilized by filtration with a filter that retains bacteria. These preparations can also be formulated with bactericides that are dissolved or dispersed in the injectable medium or by other methods known in the art.

### Combination Therapy

The compounds of the present invention can be used in combination with other drugs for the prevention or treatment of SSTR5-mediated diseases.

The compounds of the present invention can be used in combination with one or more other drugs to treat, prevent or improve diseases for which the compounds of the present invention or other drugs may be effective, where the combination of these drugs is safer or more effective than any one of the drugs alone. The other drugs can be administered simultaneously, prior to or after the administration of the compounds of the present invention via commonly used routes of administration in common doses. When the compounds of the present invention are used simultaneously with one or more other drugs, it is preferred that a unit dosage form of the pharmaceutical composition contains the other drugs and the compounds of the present invention.

However, combination therapy can also include therapies in which the compounds of the general formula herein and one or more other drugs are administered in different overlapping regimens. When used in combination with one or more other active ingredients, the compounds of the present invention and the other drugs can be used at lower doses than the doses needed when they are used alone. The other drugs include but are not limited to TGR5 agonists, GPR119 agonists, GPR40 agonists, PDE4 inhibitors, DPP4 inhibitors, SGLT2 inhibitors, metformin, insulin sensitizers, insulin and its analogs, α-glucosidase inhibitors, sulfonylurea or non-sulfonylurea insulin secretion promoters, and incretin analogs, etc.

The present invention will be further explained by examples below. It should be noted that these examples are only used to illustrate the present invention and are not intended to limit the present invention in any way. All parameters and other descriptions in the examples are based on mass unless otherwise specified. The fillers used for column chromatography separation are all silica gel unless otherwise specified. The experimental methods in the following examples that do not specify specific conditions are usually carried out under conventional conditions or under conditions recommended by the manufacturer.

Unless otherwise defined, all the professional and scientific terms used in the text have the same meanings as those familiar to those skilled in the art. In addition, any methods and materials similar to or equivalent to those described can be applied to the present invention. The preferred implementation methods and materials described in the text are for demonstration purposes only.

### EXAMPLES

### Example 1

### 4-(9-((2,6-diethoxy-4′-fluoro-[1,1′-biphenyl]-4-yl)methyl)-3-oxo-2,9-diazaspiro[5.5]undec-2-yl)benzoic acid, trifluoroacetate

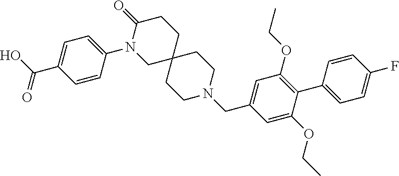 [SMILES: CCOc1cc(CN2CCC3(CCC(=O)N(c4ccc(C(=O)O)cc4)C3)CC2)cc(OCC)c1-c1ccc(F)cc1]

### Step 1: Preparation of Intermediate tert-butyl 2-(4-(methoxycarbonyl)phenyl)-3-oxo-2,9-diazaspiro[5.5]undecane-9-carboxylate (A1a)

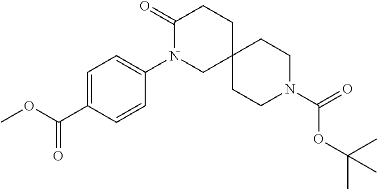 [SMILES: COC(=O)c1ccc(N2CC3(CCC2=O)CCN(C(=O)OC(C)(C)C)CC3)cc1]

tert-butyl 3-oxo-2,9-diazaspiro[5.5]undecane-9-carboxylate (1 eq), methyl p-bromobenzoate (1.5 eq), anhydrous potassium phosphate (3 eq) and Xantphos ligand (0.2 eq) were dissolved in 1,4-dioxane solution, and Pd₂(dba)₃ (0.1 eq) was added after nitrogen replacement. After nitrogen replacement was performed again, and the mixture was reacted at 100-110° C. for 3 h in a sealed tube. The reaction solution was cooled to room temperature, and then the solid in the reaction solution was filtered out. The filtrate was directly mixed with silica gel and subjected to column chromatography purification to obtain the target product with a yield of about 70%.

MS (ESI): m/z 303.3 [M−Boc+H]⁺

¹H NMR (500 MHz, Chloroform-d) δ 8.06 (d, J=8.5 Hz, 2H), 7.32 (d, J=8.6 Hz, 2H), 3.91 (s, 4H), 3.57-3.45 (m, 4H), 3.37 (ddd, J=13.9, 8.2, 3.9 Hz, 2H), 2.60 (t, J=7.2 Hz, 2H), 1.86 (t, J=7.2 Hz, 2H), 1.59 (dtq, J=21.8, 8.4, 4.2 Hz, 4H), 1.45 (s, 9H).

### Step 2: Preparation of Intermediate methyl 4-(3-oxo-2,9-diazaspiro[5.5]undec-2-yl)benzoate hydrochloride (A1b)

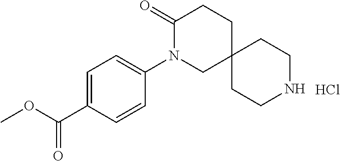 [SMILES: COC(=O)c1ccc(N2CC3(CCNCC3)CCC2=O)cc1 ; Cl]

The intermediate (A1a) obtained in the previous step was dissolved in DCM, and an excess of hydrogen chloride/dioxane solution (4 N) was added at room temperature. The reaction was carried out for 2 h at room temperature. After the reaction was complete as confirmed by TLC, the reaction solution was evaporated to dryness to obtain a solid target product, which could be directly used in the next step without purification. Yield 92%.

MS (ESI): m/z 503.3 [M+H]⁺.

### Step 3: Preparation of Intermediate 4-(chloromethyl)-2,6-diethoxy-4′-fluoro-1,1′-biphenyl (A1c)

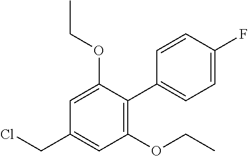 [SMILES: CCOc1cc(CCl)cc(OCC)c1-c1ccc(F)cc1]

Please refer to ACS Med. Chem. Lett. 2018, 9, 11, 1082-1087 for the preparation method.

¹H NMR (500 MHz, chloroform-d) δ 7.39-7.31 (m, 2H), 7.12-7.05 (m, 2H), 6.68 (s, 2H), 4.61 (s, 2H), 4.01 (q, J=7.0 Hz, 4H), 1.28 (t, J=6.9 Hz, 6H).

MS (ESI): m/z 309.2 [M+H]⁺.

### Step 4: Preparation of Intermediate methyl 4-(9-((2,6-diethoxy-4′-fluoro-[1,1′-biphenyl]-4-yl)methyl)-3-oxo-2,9-diazaspiro[5.5]undec-2-yl)benzoate (A1d)

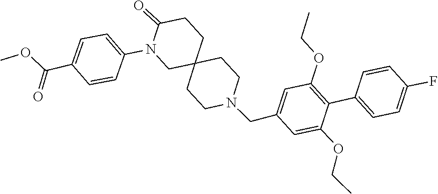 [SMILES: CCOc1cc(CN2CCC3(CCC(=O)N(c4ccc(C(=O)OC)cc4)C3)CC2)cc(OCC)c1-c1ccc(F)cc1]

The intermediate 4-(chloromethyl)-2,6-diethoxy-4′-fluoro-1,1′-biphenyl (A1c) (1 eq), the intermediate methyl 4-(3-oxo-2,9-diazaspiro[5.5]undec-2-yl)benzoate hydrochloride (A1b) (1 eq), and cesium carbonate (1.4 eq) were dissolved in acetonitrile and reacted at 60° C. for 3 h. After the reaction was complete as confirmed by TLC, the solid in the reaction solution was filtered out, the filtrate was directly mixed with silica gel and subjected to Flash column separation to obtain the target product with a yield of 72%.

MS (ESI): m/z 575.4 [M+H]⁺.

¹H NMR (500 MHz, Chloroform-d) δ 8.06 (d, J=8.6 Hz, 2H), 7.33 (ddd, J=8.8, 3.9, 1.8 Hz, 5H), 7.04 (t, J=8.9 Hz, 2H), 6.59 (s, 3H), 3.96 (q, J=7.0 Hz, 4H), 3.92 (s, 3H), 3.52 (s, 2H), 3.49 (d, J=3.6 Hz, 2H), 2.59 (t, J=7.1 Hz, 2H), 2.48 (s, 4H), 1.84 (t, J=7.2 Hz, 2H), 1.68 (s, 4H), 1.23 (t, J=7.0 Hz, 6H).

### Step 5: Preparation of Final Product A1: 4-(9-((2,6-diethoxy-4′-fluoro-[1,1′-biphenyl]-4-yl)methyl)-3-oxo-2,9-diazaspiro[5.5]undec-2-yl)benzoic acid, trifluoroacetate

The intermediate A1d (1 eq) was dissolved in a mixed solvent of 1,4-dioxane and water (volume ratio=4:1), and lithium hydroxide monohydrate (2 eq) was added and reacted at 50° C. for 12 hours. After the reaction was complete as confirmed by TLC, the reaction solution was concentrated by evaporation, and the trifluoroacetate Ale was obtained by semi-preparative liquid phase purification. Yield 65%.

In the semi-preparative liquid phase purification, the mobile phase was acetonitrile-water (containing 0.1% trifluoroacetic acid), and the elution gradient was: 0 min: 20% acetonitrile—80% water (containing 0.1% trifluoroacetic acid); 45 min: 75% acetonitrile—25% water (containing 0.1% trifluoroacetic acid)

MS (ESI): m/z 561.3 [M+H]⁺.

¹H NMR (500 MHz, DMSO-d6) δ 11.13 (s, 1H), 7.94 (d, J=8.1 Hz, 2H), 7.39 (d, J=8.1 Hz, 2H), 7.27 (dd, J=8.6, 5.8 Hz, 2H), 7.15 (t, J=8.9 Hz, 2H), 7.02 (s, 2H), 4.22 (s, 2H), 3.97 (q, J=7.0 Hz, 4H), 3.80-3.57 (m, 2H), 3.20-2.92 (m, 6H), 2.03-1.59 (m, 6H), 1.15 (t, J=6.9 Hz, 6H).

### Example 2

### 4-(5-((2,6-diethoxy-4′-fluoro-[1,1′-biphenyl]-4-yl)methyl)octahydro-1H-pyrrolo[3,2-c]pyridin-1-yl)benzoic acid, trifluoroacetate

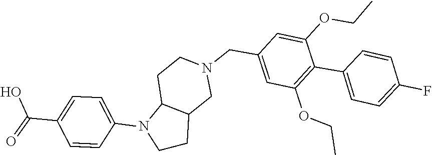 [SMILES: CCOc1cc(CN2CCC3C(CCN3c3ccc(C(=O)O)cc3)C2)cc(OCC)c1-c1ccc(F)cc1]

The preparation steps were the same as those in Example 1, except that tert-butyl 3-oxo-2,9-diazaspiro[5.5]undecane-9-carboxylate (CAS: 1251021-18-7) was replaced by octahydro-6H-pyrrolo[2,3-c]pyridine-6-carboxylic acid (CAS: 1196147-27-9) as the starting material.

MS (ESI): m/z 519.4 [M+H]⁺.

¹H NMR (500 MHz, DMSO-d6) δ 9.68 (s, 1H), 7.74 (d, J=8.5 Hz, 2H), 7.30 (dd, J=8.4, 5.6 Hz, 3H), 7.19 (d, J=8.8 Hz, 2H), 6.93 (s, 2H), 6.62 (d, J=8.6 Hz, 2H), 4.34 (d, J=38.9 Hz, 2H), 4.01 (q, J=7.1 Hz, 4H), 3.55-3.46 (m, 2H), 3.46-3.41 (m, 2H), 3.28 (d, J=7.2 Hz, 2H), 2.67-2.61 (m, 1H), 2.59-2.53 (m, 1H), 2.39-2.34 (m, 1H), 1.99 (dt, J=17.2, 7.0 Hz, 1H), 1.58 (s, 1H), 1.46 (t, J=7.2 Hz, 1H), 1.23-1.12 (m, 6H).

### Example 3

### 4-(9-((2,6-diethoxy-4′-fluoro-[1,1′-biphenyl]-4-yl)methyl)-2-oxo-3,9-diazaspiro[5.5]undec-3-yl)benzoic acid, trifluoroacetate

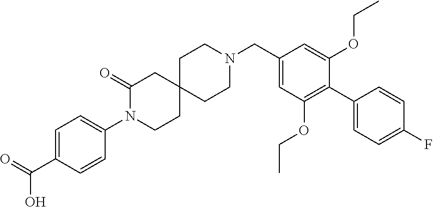 [SMILES: CCOc1cc(CN2CCC3(CC2)CCN(c2ccc(C(=O)O)cc2)C(=O)C3)cc(OCC)c1-c1ccc(F)cc1]

### Step 1: Preparation of Intermediate 9-(2,6-diethoxy-4′-fluoro-[1,1′-biphenyl]-4-yl)methyl)-3,9-diazaspiro[5.5]-2-undecanone (A3a)

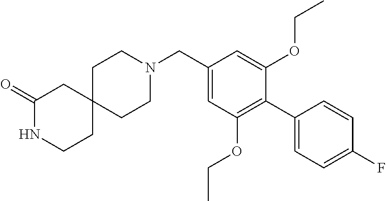 [SMILES: CCOc1cc(CN2CCC3(CCNC(=O)C3)CC2)cc(OCC)c1-c1ccc(F)cc1]

3,9-diazaspiro[5.5]-2-undecanone (1 eq), intermediate A1c (1 eq) and cesium carbonate (1.4 eq) were dissolved in acetonitrile and reacted at 60° C. for 3 h. After the reaction was complete as confirmed by TLC, the solid in the reaction solution was filtered out, the filtrate was directly mixed with the silica gel and subjected to Flash column separation to obtain the target product with a yield of 77%.

MS (ESI): m/z 441.2 [M+H]⁺.

¹H NMR (500 MHz, Chloroform-d) δ 7.34 (dd, J=8.6, 5.8 Hz, 2H), 7.04 (t, J=8.8 Hz, 2H), 6.60 (s, 2H), 6.15 (s, 1H), 3.96 (q, J=7.0 Hz, 4H), 3.52 (s, 2H), 3.37-3.31 (m, 2H), 2.64-2.35 (m, 4H), 2.27 (s, 2H), 1.69 (t, J=6.3 Hz, 2H), 1.57 (s, 4H), 1.24 (t, J=7.0 Hz, 6H).

### Step 2: Preparation of Intermediate methyl 4-(9-((2,6-diethoxy-4′-fluoro-[1,1′-biphenyl]-4-yl)methyl)-2-oxo-3,9-diazaspiro[5.5]undecane-3-yl)benzoate (A3b)

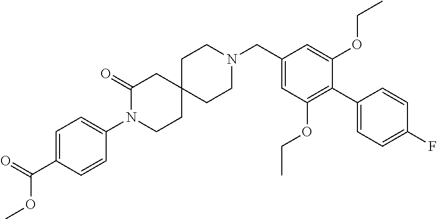 [SMILES: CCOc1cc(CN2CCC3(CC2)CCN(c2ccc(C(=O)OC)cc2)C(=O)C3)cc(OCC)c1-c1ccc(F)cc1]

9-(2,6-diethoxy-4′-fluoro-[1,1′-biphenyl]-4-yl)methyl)-3,9-diazaspiro[5.5]-2-undecanone (A3a) (1 eq), methyl p-bromobenzoate (1.5 eq), anhydrous potassium phosphate (3 eq) and Xantphos ligand (0.2 eq) were dissolved in 1,4-dioxane solution, and Pd₂(dba)₃ (0.1 eq) was added after nitrogen replacement. After nitrogen replacement was performed again, and the mixture was reacted in a sealed tube at 100-110° C. for 3 h. The reaction solution was cooled to room temperature, and then the solid in the reaction solution was filtered out, the filtrate was directly mixed with silica gel and subjected to column chromatography purification to obtain the target product. Yield 60%.

MS (ESI): m/z 575.3 [M+H]⁺∘

¹H NMR (500 MHz, Chloroform-d) δ 8.09-8.03 (m, 2H), 7.38-7.31 (m, 4H), 7.05 (t, J=8.9 Hz, 2H), 6.60 (s, 2H), 3.97 (q, J=7.0 Hz, 4H), 3.91 (s, 3H), 3.73-3.67 (m, 2H), 3.53 (s, 2H), 2.63-2.53 (m, 2H), 2.53-2.41 (m, 4H), 1.88 (t, J=6.3 Hz, 2H), 1.70-1.62 (m, 4H), 1.25 (t, J=7.0 Hz, 6H).

### Step 3: Preparation of Final Product A3c: 4-(9-((2,6-diethoxy-4′-fluoro-[1,1′-biphenyl]-4-yl)methyl)-2-oxo-3,9-diazaspiro[5.5]undec-3-yl)benzoic acid, trifluoroacetate

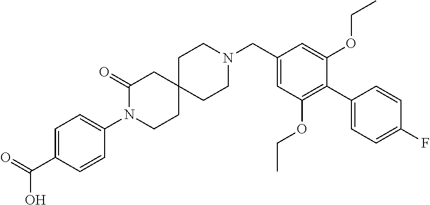 [SMILES: CCOc1cc(CN2CCC3(CC2)CCN(c2ccc(C(=O)O)cc2)C(=O)C3)cc(OCC)c1-c1ccc(F)cc1]

The intermediate A3b (1 eq) was dissolved in a mixed solvent of 1,4-dioxane and water (volume ratio=4:1), and lithium hydroxide monohydrate (2 eq) was added and reacted at 50° C. for 12 hours. After the reaction was complete as confirmed by TLC, the reaction solution was concentrated by evaporation, and the trifluoroacetate A3c was obtained by semi-preparative liquid phase purification. Yield 68%.

MS (ESI): m/z 561.3 [M+H]⁺.

¹H NMR (500 MHz, DMSO-d6) δ 10.06 (s, 1H), 7.95 (d, J=8.2 Hz, 2H), 7.45 (d, J=8.2 Hz, 2H), 7.30 (dd, J=8.5, 5.6 Hz, 2H), 7.18 (t, J=8.8 Hz, 2H), 6.88 (s, 2H), 4.32 (s, 2H), 4.00 (q, J=6.9 Hz, 4H), 3.71 (s, 2H), 3.34-3.07 (m, 6H), 2.11-1.65 (m, 6H), 1.18 (t, J=6.9 Hz, 6H).

### Example 4

### 4-(3-(1-((2,6-diethoxy-4′-fluoro-[1,1′-biphenyl]-4-yl)methyl)piperidin-4-yl)azetidin-1-yl)benzoic acid, trifluoroacetate

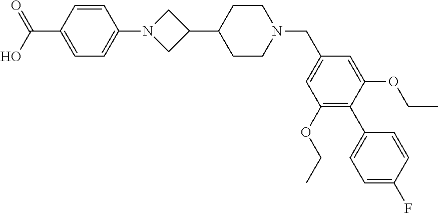 [SMILES: CCOc1cc(CN2CCC(C3CN(c4ccc(C(=O)O)cc4)C3)CC2)cc(OCC)c1-c1ccc(F)cc1]

The preparation steps were the same as those in Example 1, except that tert-butyl 3-oxo-2,9-diazaspiro[5.5]undecane-9-carboxylate (CAS: 1251021-18-7) was replaced by tert-butyl 4-(azetidin-3-yl)piperidin-1-carboxylate (CAS: 1314703-47-3) as the starting material.

MS (ESI): m/z 533.2 [M+H]⁺.

¹H NMR (500 MHz, DMSO-d6) δ 7.74 (d, J=8.3 Hz, 2H), 7.29 (dd, J=8.3, 5.7 Hz, 2H), 7.15 (t, J=8.7 Hz, 2H), 6.68 (s, 2H), 6.39 (dd, J=8.7, 2.4 Hz, 2H), 3.96 (p, J=7.8, 7.0 Hz, 6H), 3.62 (t, J=6.7 Hz, 2H), 3.56-3.47 (m, 2H), 2.98-2.84 (m, 2H), 2.11-1.89 (m, 2H), 1.68 (d, J=12.5 Hz, 2H), 1.48 (dd, J=17.1, 9.0 Hz, 1H), 1.24 (s, 3H), 1.15 (t, J=7.0 Hz, 6H).

### Example 5

### 4-(9-((2,6-diethoxy-4′-fluoro-[1,1′-biphenyl]-4-yl)methyl)-1-oxo-4,9-diazaspiro[5.5]undec-4-yl)benzoic acid, trifluoroacetate

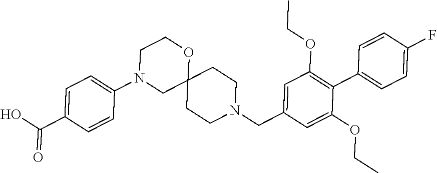 [SMILES: CCOc1cc(CN2CCC3(CC2)CN(c2ccc(C(=O)O)cc2)CCO3)cc(OCC)c1-c1ccc(F)cc1]

The preparation steps were the same as those in Example 1, except that tert-butyl 3-oxo-2,9-diazaspiro[5.5]undecane-9-carboxylate (CAS: 1251021-18-7) was replaced by tert-butyl 1-oxa-4,9-diazaspiro[5.5]undecane-9-carboxylate (CAS: 930785-40-3).

MS (ESI): m/z 549.2 [M+H]⁺.

¹H NMR (500 MHz, DMSO-d6) δ 9.97 (s, 1H), 7.77 (d, J=8.7 Hz, 2H), 7.28 (dd, J=8.7, 5.8 Hz, 2H), 7.16 (t, J=8.9 Hz, 2H), 6.97 (d, J=8.7 Hz, 2H), 6.90 (s, 2H), 4.30 (s, 2H), 3.97 (q, J=6.9 Hz, 4H), 3.84-3.74 (m, 2H), 3.32-3.23 (m, 4H), 3.17-3.03 (m, 2H), 2.16 (d, J=14.3 Hz, 2H), 1.77 (td, J=14.5, 4.1 Hz, 2H), 1.16 (t, J=7.0 Hz, 6H).

### Example 6

### 4-((2-((2,6-diethoxy-4′-fluoro-[1,1′-biphenyl]-4-yl)methyl)-2-azaspiro[3.3]heptane-6-yl)amino)benzoic acid, trifluoroacetate

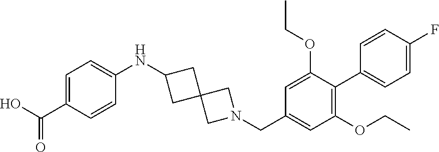 [SMILES: CCOc1cc(CN2CC3(CC(Nc4ccc(C(=O)O)cc4)C3)C2)cc(OCC)c1-c1ccc(F)cc1]

### Step 1: Preparation of Intermediate tert-butyl (2-((2,6-diethoxy-4′-fluoro-[1,1′-biphenyl]-4-yl)methyl)-2-azaspiro[3.3]heptane-6-yl)carbamate (A6a)

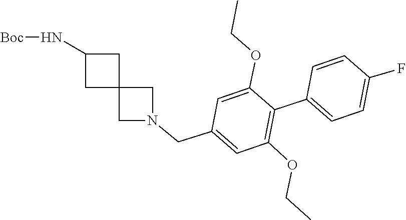 [SMILES: CCOc1cc(CN2CC3(CC(NC(=O)OC(C)(C)C)C3)C2)cc(OCC)c1-c1ccc(F)cc1]

Tert-butyl (2-azaspiro[3.3]heptane-6-yl)carbamate (1 eq), intermediate A1c (1 eq) and cesium carbonate (1.4 eq) were dissolved in acetonitrile and reacted at 60° C. for 3 h. After the reaction was complete as confirmed by TLC, the solid in the reaction solution was filtered out, the filtrate was directly mixed with the silica gel, and subjected to Flash column separation to obtain the target product (A6a) with a yield of 81%.

MS (ESI): m/z 385.3 [M−Boc+H]⁺.

¹H NMR (500 MHz, Chloroform-d) δ 7.32 (dd, J=8.7, 5.7 Hz, 2H), 7.03 (t, J=8.8 Hz, 2H), 6.54 (s, 2H), 3.96 (q, J=7.0 Hz, 4H), 3.59 (s, 2H), 3.34 (s, 2H), 3.24 (s, 2H), 2.56 (t, J=9.9 Hz, 2H), 1.97 (td, J=8.9, 2.9 Hz, 2H), 1.42 (s, 9H), 1.23 (t, J=7.0 Hz, 6H).

### Step 2: Preparation of Intermediate 2-((2,6-diethoxy-4′-fluoro-[1,1′-biphenyl]-4-yl)methyl)-2-azaspiro[3.3]heptane-6-amine hydrochloride (A6b)

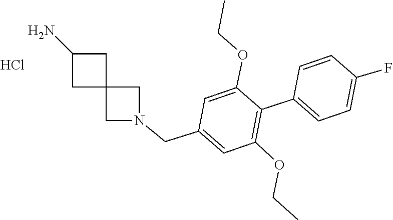 [SMILES: Cl ; CCOc1cc(CN2CC3(CC(N)C3)C2)cc(OCC)c1-c1ccc(F)cc1]

The intermediate A6a obtained in the previous step was dissolved in dichloromethane, and an excess of hydrogen chloride/dioxane solution (4N) was added to react at room temperature for 2 h. After the reaction was complete as confirmed by TLC, the reaction solution was evaporated to dryness to obtain a solid target product (A6b), which could be directly used in the next step without purification. Yield 90%.

MS (ESI): m/z 385.3 [M+H]⁺.

### Step 3: Preparation of Intermediate methyl 4-((2-((2,6-diethoxy-4′-fluoro-[1,1′-biphenyl]-4-yl)methyl)-2-azaspiro[3.3]heptane-6-yl)amino)benzoate (A6c)

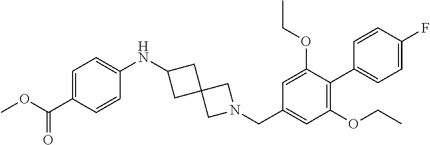 [SMILES: CCOc1cc(CN2CC3(CC(Nc4ccc(C(=O)OC)cc4)C3)C2)cc(OCC)c1-c1ccc(F)cc1]

2-((2,6-diethoxy-4′-fluoro-[1,1′-biphenyl]-4-yl)methyl)-2-azaspiro[3.3]heptane-6-amine hydrochloride (A6b) (1 eq), methyl p-bromobenzoate (1.5 eq), anhydrous potassium phosphate (3 eq) and Xantphos ligand (0.2 eq) were dissolved in 1,4-dioxane solution, and Pd₂(dba)₃ (0.1 eq) was added after nitrogen replacement. After nitrogen replacement was performed again, and the mixture was reacted in a sealed tube at 100-110° C. for 3 h. The reaction solution was cooled to room temperature, and then the solid in the reaction solution was filtered out. The filtrate was directly mixed with silica gel and subjected to Flash column purification to obtain the target product (A6c) with a yield of 45%.

MS (ESI): m/z 519.3 [M+H]⁺.

¹H NMR (500 MHz, Chloroform-d) δ 7.88 (d, J=8.9 Hz, 2H), 7.37-7.28 (m, 2H), 7.11-6.99 (m, 2H), 6.65-6.59 (m, 4H), 3.97 (q, J=7.0 Hz, 4H), 3.85 (s, 3H), 3.83 (s, 2H), 3.33 (s, 2H), 2.97 (s, 2H), 1.88 (dt, J=4.0, 1.8 Hz, 2H), 1.56 (dd, J=4.4, 1.9 Hz, 2H), 1.25 (t, J=7.0 Hz, 6H).

### Step 4: Preparation of the Final Product 4-((2-((2,6-diethoxy-4′-fluoro-[1,1′-biphenyl]-4-yl)methyl)-2-azaspiro[3.3]heptane-6-yl)amino)benzoic acid (A6d)

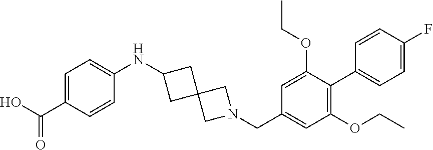 [SMILES: CCOc1cc(CN2CC3(CC(Nc4ccc(C(=O)O)cc4)C3)C2)cc(OCC)c1-c1ccc(F)cc1]

The intermediate A6c (1 eq) was dissolved in a mixed solvent of 1,4-dioxane and water (volume ratio=4:1), and lithium hydroxide monohydrate (2 eq) was added and reacted at 50° C. for 12 hours. After the reaction was complete as confirmed by TLC, the reaction solution was concentrated by evaporation. A6d was obtained by semi-preparative liquid phase purification with a yield of 66%.

MS (ESI): m/z 505.2 [M+H]⁺.

¹H NMR (500 MHz, DMSO-d6) δ 12.16 (s, 1H), 9.24 (s, 2H), 7.74 (d, J=8.4 Hz, 2H), 7.28 (dd, J=8.4, 5.6 Hz, 2H), 7.17 (t, J=8.7 Hz, 2H), 6.93 (s, 2H), 6.70 (d, J=8.5 Hz, 2H), 4.56 (s, 1H), 4.20 (s, 2H), 3.99 (q, J=7.0 Hz, 4H), 3.34 (s, 4H), 2.04 (d, J=4.5 Hz, 2H), 1.50 (d, J=4.4 Hz, 2H), 1.18 (t, J=6.9 Hz, 6H).

### Example 7

### 4-(2-((2,6-diethoxy-4′-fluoro-[1,1′-biphenyl]-4-yl)methyl)-7-oxo-2,6-diazaspiro[3.4]octane-6-yl)benzoic acid, trifluoroacetate

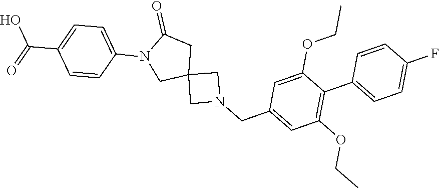 [SMILES: CCOc1cc(CN2CC3(CC(=O)N(c4ccc(C(=O)O)cc4)C3)C2)cc(OCC)c1-c1ccc(F)cc1]

The preparation method was the same as that in Example 1, except that tert-butyl 3-oxo-2,9-diazaspiro[5.5]undecane-9-carboxylate (CAS: 1251021-18-7) was replaced by tert-butyl 7-oxo-2,6-diazaspiro[3,4]octane-2-carboxylate (CAS: 1234616-51-3).

MS (ESI): m/z 519.2 [M+H]⁺.

¹H NMR (500 MHz, DMSO-d6) δ 11.97 (s, 1H), 7.97 (d, J=8.5 Hz, 2H), 7.73 (d, J=8.5 Hz, 2H), 7.28 (dd, J=8.6, 5.8 Hz, 2H), 7.21-7.09 (m, 2H), 7.03-6.93 (m, 2H), 4.35 (s, 2H), 4.29-4.21 (m, 2H), 4.22-4.07 (m, 4H), 4.01 (q, J=7.0 Hz, 4H), 3.03 (s, 2H), 1.18 (t, J=7.0 Hz, 6H).

### Example 8

### 4-(2-(2-cyclopropyl-5-ethoxy-4-methylbenzyl)-7-oxo-2,6-diazaspiro[3.4]octane-6-yl)benzoic acid, trifluoroacetate

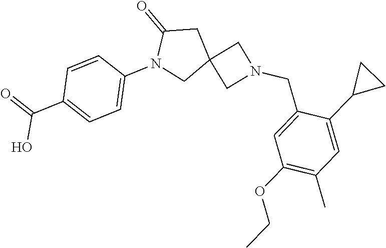 [SMILES: CCOc1cc(CN2CC3(CC(=O)N(c4ccc(C(=O)O)cc4)C3)C2)c(C2CC2)cc1C]

### Step 1: Preparation of Intermediate tert-butyl 6-(4-(methoxycarbonyl)phenyl)-7-oxo-2,6-diazaspiro[3.4]octane-2-carboxylate (A8a)

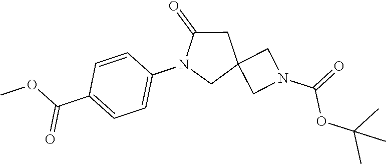 [SMILES: COC(=O)c1ccc(N2CC3(CC2=O)CN(C(=O)OC(C)(C)C)C3)cc1]

tert-butyl 7-Oxo-2,6-diazaspiro[3.4]octane-2-carboxylate (1 eq), methyl p-bromobenzoate (1.5 eq), anhydrous potassium phosphate (3 eq) and Xantphos ligand (0.2 eq) were dissolved in 1,4-dioxane solution, and Pd₂(dba)₃ (0.1 eq) was added after nitrogen replacement. After nitrogen replacement was performed again, and the mixture was reacted in a sealed tube at 100-110° C. for 3 h. The reaction solution was cooled to room temperature, and then the solid in the reaction solution was filtered out. The filtrate was directly mixed with silica gel and purified by a Flash column to obtain the target product (A8a) with a yield of 84%.

MS (ESI): m/z 261.1 [M−BOC+H]⁺.

¹H NMR (500 MHz, Chloroform-d) δ 8.04 (d, J=8.8 Hz, 2H), 7.67 (d, J=8.8 Hz, 2H), 4.04 (s, 2H), 4.02-3.96 (m, 4H), 3.90 (s, 3H), 2.87 (s, 2H), 1.44 (s, 9H).

### Step 2: Preparation of Intermediate methyl 4-(7-oxo-2,6-diazaspiro[3.4]octan-6-yl)benzoate hydrochloride (A8b)

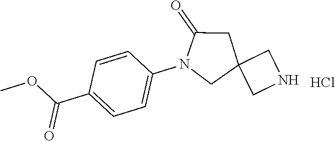 [SMILES: COC(=O)c1ccc(N2CC3(CNC3)CC2=O)cc1 ; Cl]

The intermediate A8a obtained in the previous step was dissolved in dichloromethane, and an excess of hydrogen chloride/dioxane solution (4N) was added to react at room temperature for 2 h. After the reaction was complete as confirmed by TLC, the reaction solution was evaporated to obtain the solid target product (A8b), which could be directly used in the next step without purification. Yield 93%.

MS (ESI): m/z 261.1 [M+H]⁺

### Step 3: Preparation of Intermediate 2-cyclopropyl-5-ethoxy-4-methylbenzoic acid ethyl ester (A8c)

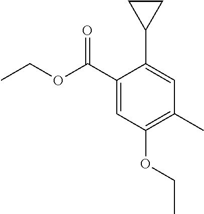 [SMILES: CCOC(=O)c1cc(OCC)c(C)cc1C1CC1]

Ethyl 2-bromo-5-ethoxy-4-methylbenzoate (1eq), cyclopropylboronic acid (1.5eq), and cesium carbonate (3eq) were dissolved in 1,4-dioxane, and PdCl₂(dppf) (0.1eq) was added after nitrogen replacement. After nitrogen replacement was performed again, the reaction was carried out at 100° C. in a sealed tube for 3 h. The reaction was stopped, and the solid in the reaction solution was filtered out after the reaction solution was cooled to room temperature. The filtrate was directly mixed with silica gel and purified by Flash column. The target product A8c was obtained with a yield of 85%.

MS (ESI): m/z 249.1 [M+H]⁺.

¹H NMR (500 MHz, Chloroform-d) δ 7.25 (s, 1H), 6.81 (t, J=0.8 Hz, 1H), 4.37 (q, J=7.1 Hz, 2H), 4.04 (q, J=7.0 Hz, 2H), 2.59-2.44 (m, 1H), 2.20 (s, 3H), 1.40 (m, 6H), 0.94-0.88 (m, 2H), 0.63-0.58 (m, 2H).

### Step 4: Preparation of Intermediate 2-cyclopropyl-5-ethoxy-4-methylbenzyl alcohol (A8d)

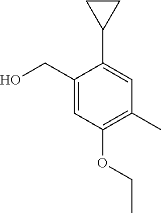

The intermediate ethyl 2-cyclopropyl-5-ethoxy-4-methylbenzoate (A8c) (1eq) was dissolved in ultra-dry tetrahydrofuran, and lithium aluminum tetrahydride (1.1eq) was added in batches under an ice bath and reacted for 2 h. After the reaction was complete as confirmed by TLC, the reaction was stopped and quenched by slowly adding 0.5M sodium hydroxide aqueous solution. The solid in the reaction solution was filtered out with diatomite, and the filter cake was repeatedly rinsed with ethyl acetate. After the filtrate was evaporated to dryness, the resultant was re-dissolved in ethyl acetate and mixed with silica gel, and subjected to Flash column separation and purification to obtain the target product with a yield of 89%.

¹H NMR (500 MHz, Chloroform-d) δ 6.90 (s, 1H), 6.84 (s, 1H), 4.88 (d, J=5.7 Hz, 2H), 4.07 (q, J=7.0 Hz, 2H), 2.21 (s, 3H), 1.93 (m, 1H), 1.44 (t, J=7.0 Hz, 3H), 0.95-0.88 (m, 2H), 0.67-0.61 (m, 2H).

### Step 5: Synthesis of Intermediate 1-(chloromethyl)-2-cyclopropyl-5-ethoxy-4-methylbenzene (A8e)

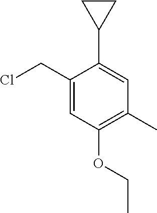

The intermediate 2-cyclopropyl-5-ethoxy-4-methylbenzyl alcohol (A8d) was dissolved in dichloromethane, and an excess of thionyl chloride was added under ice bath and reacted for 3 hours. After the reaction was complete as confirmed by TLC, the reaction solution was evaporated to dryness (sodium hydroxide aqueous solution was added to the tail bottle of the rotary evaporator). The resultant was then re-dissolved in dichloromethane, and mixed with silica gel and separated by Flash column to obtain the target product A8e with a yield of 92%.

¹H NMR (500 MHz, Chloroform-d) δ 6.84 (t, J=0.8 Hz, 1H), 6.79 (s, 1H), 4.79 (s, 2H), 4.03 (q, J=7.0 Hz, 2H), 2.18 (d, J=0.7 Hz, 3H), 1.98 (ttd, J=8.5, 5.4, 0.8 Hz, 1H), 1.41 (t, J=6.9 Hz, 3H), 0.96-0.90 (m, 2H), 0.66-0.61 (m, 2H).

### Step 6: Preparation of Intermediate methyl 4-(2-(2-cyclopropyl-5-ethoxy-4-methylbenzyl)-7-oxo-2,6-diazaspiro[3.4]octan-6-yl)benzoate (A8f)

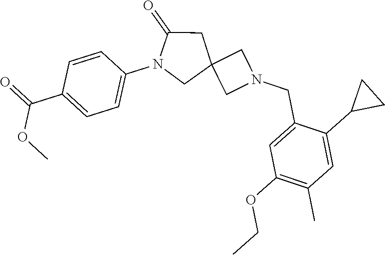 [SMILES: CCOc1cc(CN2CC3(CC(=O)N(c4ccc(C(=O)OC)cc4)C3)C2)c(C2CC2)cc1C]

The intermediate methyl 4-(7-oxo-2,6-diazaspiro[3.4]octan-6-yl)benzoate hydrochloride (A8b) (1eq), the intermediate 1-(chloromethyl)-2-cyclopropyl-5-ethoxy-4-methylbenzene (A8e) (1eq), and potassium carbonate (1.5eq) were dissolved in DMF and reacted at room temperature overnight. After the reaction was complete as confirmed by TLC, the reaction solution was extracted three times with ethyl acetate-water system. The ethyl acetate layers were combined and washed three times with saturated sodium chloride aqueous solution. The ethyl acetate layer was dried over anhydrous sodium sulfate, mixed with silica gel and then separated and purified by Flash column to obtain the target product A8f with a yield of 70%.

MS (ESI): m/z 449.2 [M+H]⁺.

¹H NMR (600 MHz, Chloroform-d) δ 8.04 (d, J=8.9 Hz, 2H), 7.72 (d, J=8.9 Hz, 2H), 6.79 (s, 2H), 4.08 (s, 2H), 4.03 (q, J=7.0 Hz, 2H), 3.91 (s, 3H), 3.78 (s, 2H), 3.37 (d, J=7.4 Hz, 2H), 3.30 (d, J=7.5 Hz, 2H), 2.85 (s, 2H), 2.16 (s, 3H), 1.88 (tt, J=8.4, 5.4 Hz, 1H), 1.41 (t, J=7.0 Hz, 3H), 0.90-0.82 (m, 2H), 0.60-0.55 (m, 2H).

### Step 7: Preparation of the Final Intermediate 4-(2-(2-cyclopropyl-5-ethoxy-4-methylbenzyl)-7-oxo-2,6-diazaspiro[3.4]octan-6-yl)benzoic acid

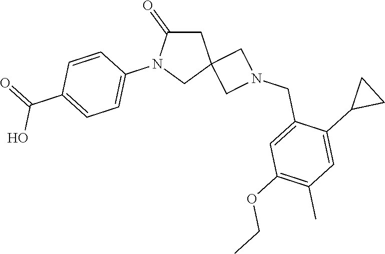 [SMILES: CCOc1cc(CN2CC3(CC(=O)N(c4ccc(C(=O)O)cc4)C3)C2)c(C2CC2)cc1C]

The intermediate methyl 4-(2-(2-cyclopropyl-5-ethoxy-4-methylbenzyl)-7-oxo-2,6-diazaspiro[3.4]octan-6-yl)benzoate A8f (1 eq) was dissolved in a mixed solvent of methanol, tetrahydrofuran and water (volume ratio=3:3:1), and lithium hydroxide monohydrate (2 eq) was added. The reaction was carried out at 50° C. for 12 hours. After the reaction was complete as confirmed by TLC, the reaction solution was concentrated by evaporation, and purified by semi-preparative liquid phase to obtain the final intermediate with a yield of 70%.

MS (ESI): m/z 434.2 [M+H]⁺.

¹H NMR (600 MHz, DMSO-d6) δ 7.93 (d, J=8.8 Hz, 2H), 7.77 (d, J=8.8 Hz, 2H), 6.83 (s, 1H), 6.71 (s, 1H), 4.07 (s, 2H), 3.99 (q, J=6.9 Hz, 2H), 3.73 (s, 2H), 3.34 (d, J=6.9 Hz, 2H), 3.30 (d, J=6.8 Hz, 2H), 2.83 (s, 2H), 2.06 (s, 3H), 1.91 (m, 1H), 1.31 (t, J=6.9 Hz, 3H), 0.86-0.80 (m, 2H), 0.54-0.50 (m, 2H).

### Example 9

### 4-(2-(2-cyclopropyl-5-ethoxybenzyl)-7-oxo-2,6-diazaspiro[3.4]octyl-6-yl)benzoic acid, trifluoroacetate

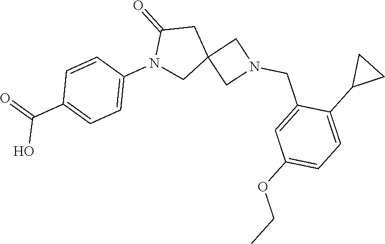 [SMILES: CCOc1ccc(C2CC2)c(CN2CC3(CC(=O)N(c4ccc(C(=O)O)cc4)C3)C2)c1]

The synthesis steps were the same as those in Example 8, except that ethyl 2-bromo-5-ethoxy-4-methylbenzoate was replaced by methyl 2-bromo-5-ethoxybenzoate (CAS: 765944-34-1).

MS (ESI): m/z 421.2 [M+H]⁺.

### Example 10

### 4-(7-((2,6-diethoxy-4′-fluoro-[1,1′-biphenyl]-4-yl)methyl)-2,7-diazaspiro[3.5]nonan-2-yl)benzoic acid, trifluoroacetate

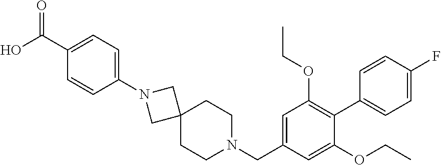 [SMILES: CCOc1cc(CN2CCC3(CC2)CN(c2ccc(C(=O)O)cc2)C3)cc(OCC)c1-c1ccc(F)cc1]

### Step 1: Preparation of Intermediate tert-butyl 2-(4-(methoxycarbonyl)phenyl)-2,7-diazaspiro[3.5]nonane-7-carboxylate (A10a)

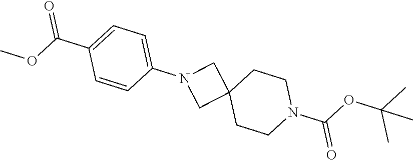 [SMILES: COC(=O)c1ccc(N2CC3(CCN(C(=O)OC(C)(C)C)CC3)C2)cc1]

tert-butyl 2,7-diazaspiro[3.5]nonane-7-carboxylate (1 eq), methyl p-bromobenzoate (1.5 eq), anhydrous potassium phosphate (3 eq) and Xantphos ligand (0.2 eq) were dissolved in 1,4-dioxane solution, and Pd₂(dba)₃ (0.1 eq) was added after nitrogen replacement. After nitrogen replacement was performed again, and the mixture was reacted at 100-110° C. for 3 h in a sealed tube. The reaction solution was cooled to room temperature, and then the solid in the reaction solution was filtered out. The filtrate was directly mixed with silica gel and subjected to column chromatography purification to obtain the target product (A10a).

MS (ESI): m/z 261.1 [M−Boc+H]⁺

¹H NMR (500 MHz, Chloroform-d) δ 7.89 (d, J=8.8 Hz, 2H), 6.36 (d, J=8.8 Hz, 2H), 3.85 (s, 3H), 3.70 (s, 4H), 3.43-3.38 (m, 4H), 1.81-1.74 (m, 4H), 1.46 (s, 9H).

### Step 2: Preparation of Intermediate methyl 4-(2,7-diazaspiro[3.5]nonan-2-yl)benzoate trifluoroacetate (A10b)

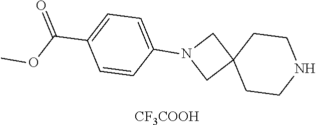 [SMILES: COC(=O)c1ccc(N2CC3(CCNCC3)C2)cc1 ; "CF3COOH"]

The intermediate A10a obtained in the previous step was dissolved in dichloromethane, and an excess of trifluoroacetic acid was added under stirring. The mixture was stirred at room temperature for 30 min. After the reaction was complete as confirmed by TLC, the reaction solution was evaporated to dryness to obtain the intermediate A10b.

MS (ESI): m/z 261.1 [M+H]⁺

### Step 3: Preparation of Intermediate methyl 4-(7-((2,6-diethoxy-4′-fluoro-[1,1′-biphenyl]-4-yl)methyl)-2,7-diazaspiro[3.5]nonan-2-yl)benzoate (A10c)

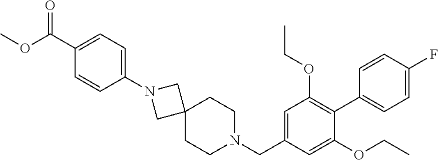 [SMILES: CCOc1cc(CN2CCC3(CC2)CN(c2ccc(C(=O)OC)cc2)C3)cc(OCC)c1-c1ccc(F)cc1]

The intermediate A10b (1 eq), intermediate A1c (1 eq), and cesium carbonate (1.4 eq) were dissolved in acetonitrile and reacted at 60° C. for 3 h. After the reaction was complete as confirmed by TLC, the solid in the reaction solution was filtered out, and the filtrate was directly mixed with silica gel, and separated by Flash column to obtain the target product A10c.

MS (ESI): m/z 532.2 [M+H]⁺.

¹H NMR (500 MHz, Chloroform-d) δ 7.88 (d, J=8.7 Hz, 2H), 7.34 (dd, J=8.7, 5.7 Hz, 2H), 7.05 (t, J=8.8 Hz, 2H), 6.61 (s, 2H), 6.36 (d, J=8.8 Hz, 2H), 3.97 (q, J=7.0 Hz, 4H), 3.85 (s, 3H), 3.68 (s, 4H), 3.48 (s, 2H), 2.44 (s, 4H), 1.87 (s, 4H), 1.25 (t, J=7.0 Hz, 6H).

### Step 4: Preparation of the Final Product 4-(7-((2,6-diethoxy-4′-fluoro-[1,1′-biphenyl]-4-yl)methyl)-2,7-diazaspiro[3.5]nonan-2-yl)benzoic acid

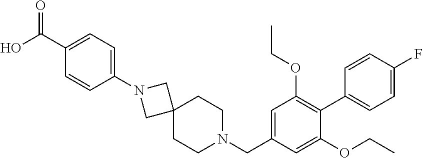 [SMILES: CCOc1cc(CN2CCC3(CC2)CN(c2ccc(C(=O)O)cc2)C3)cc(OCC)c1-c1ccc(F)cc1]

The intermediate A10c: methyl 4-(7-((2,6-diethoxy-4′-fluoro-[1,1′-biphenyl]-4-yl)methyl)-2,7-diazaspiro[3.5]nonan-2-yl)benzoate (1eq) was dissolved in a mixed solvent of methanol, tetrahydrofuran and water (volume ratio=3:3:1), and lithium hydroxide monohydrate (2eq) was added. The reaction was carried out at 50° C. for 12 hours. After the reaction was complete as confirmed by TLC, the reaction solution was concentrated by evaporation, and purified by semi-preparative liquid phase to obtain the target product.

MS (ESI): m/z 519.2 [M+H]⁺.

¹H NMR (600 MHz, DMSO-d6) δ 12.12 (s, 1H), 7.76 (d, J=8.6 Hz, 2H), 7.30 (dd, J=8.4, 6.0 Hz, 2H), 7.17 (t, J=8.9 Hz, 2H), 7.07 (s, 2H), 6.42 (d, J=8.7 Hz, 2H), 4.26 (d, J=5.0 Hz, 2H), 4.01 (q, J=6.9 Hz, 4H), 3.78 (s, 2H), 3.69 (s, 2H), 3.29 (d, J=12.0 Hz, 2H), 2.98 (q, J=9.8 Hz, 2H), 2.21-2.13 (m, 2H), 2.13-2.07 (m, 2H), 1.18 (t, J=6.9 Hz, 6H).

### Example 11

### N-(7-((2,6-diethoxy-4′-fluoro-[1,1′-biphenyl]-4-yl)methyl)-7-azaspiro[3.5]nonan-2-yl)-4-(trifluoromethoxy)benzenesulfonamide, trifluoroacetate

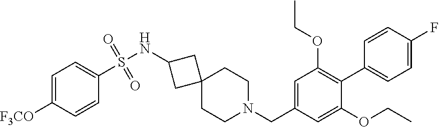 [SMILES: CCOc1cc(CN2CCC3(CC2)CC(NS(=O)(=O)c2ccc(OC(F)(F)F)cc2)C3)cc(OCC)c1-c1ccc(F)cc1]

### Step 1: Preparation of Intermediate tert-butyl 2-(4-(trifluoromethoxy)phenyl)sulfonamido)-7-azaspiro[3.5]nonane-7-carboxylate (B11a)

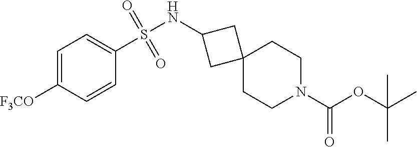 [SMILES: CC(C)(C)OC(=O)N1CCC2(CC1)CC(NS(=O)(=O)c1ccc(OC(F)(F)F)cc1)C2]

MS (ESI): m/z 365.1 [M−Boc+H]⁺.

4-Trifluoromethoxybenzenesulfonyl chloride (1 eq) and tert-butyl 2-amino-7-azaspiro[3.5]nonane-7-carboxylate (1 eq) were dissolved in dichloromethane and reacted at room temperature for 2 h. After the reaction was complete as confirmed by TLC, the reaction solution was directly mixed with silica gel, seperated and purified by Flash column to obtain the target product B11a with a yield of 93%.

¹H NMR (500 MHz, Chloroform-d) δ 7.92-7.88 (m, 2H), 7.37-7.32 (m, 2H), 4.71 (d, J=8.3 Hz, 1H), 3.87-3.77 (m, 1H), 3.29 (t, J=5.7 Hz, 2H), 3.25-3.19 (m, 2H), 2.17 (ddt, J=12.1, 7.9, 1.7 Hz, 2H), 1.57-1.54 (m, 2H), 1.48 (t, J=5.6 Hz, 2H), 1.43 (s, 11H).

### Step 2: Preparation of Intermediate N-(7-azaspiro[3.5]nonan-2-yl)-4-(trifluoromethoxy)benzenesulfonamide hydrochloride (B11b)

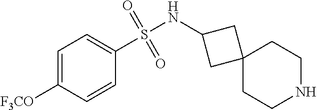 [SMILES: O=S(=O)(NC1CC2(CCNCC2)C1)c1ccc(OC(F)(F)F)cc1]

The intermediate B11a obtained in the previous step was dissolved in dichloromethane, and an excess of hydrogen chloride/dioxane solution (4N) was added to react at room temperature for 2 h. After the reaction was complete as confirmed by TLC, the reaction solution was evaporated to dryness to obtain a solid target product (B11b), which could be directly used in the next reaction without purification. Yield 95%.

MS (ESI): m/z 365.1 [M+H]⁺

### Step 3: Preparation of the Final Product N-(7-((2,6-diethoxy-4′-fluoro-[1,1′-biphenyl]-4-yl)methyl)-7-azaspiro[3.5]nonan-2-yl)-4-(trifluoromethoxy)benzenesulfonamide (B11c)

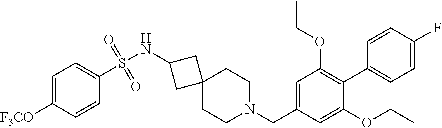 [SMILES: CCOc1cc(CN2CCC3(CC2)CC(NS(=O)(=O)c2ccc(OC(F)(F)F)cc2)C3)cc(OCC)c1-c1ccc(F)cc1]

The intermediate N-(7-azaspiro[3.5]nonan-2-yl)-4-(trifluoromethoxy)benzenesulfonamide hydrochloride B11b (1 eq), the intermediate 4-(chloromethyl)-2,6-diethoxy-4′-fluoro-1,1′-biphenyl (A1c) (1 eq), and potassium carbonate (3 eq) were dissolved in DMF and reacted at room temperature overnight. After the reaction was complete as confirmed by TLC, the reaction solution was extracted three times with an ethyl acetate-water system. The ethyl acetate layers were combined and then washed three times with a saturated sodium chloride aqueous solution. The ethyl acetate layer was dried over anhydrous sodium sulfate and purified by preparative liquid phase (mobile phase: MeCN—H₂O system, containing 0.1% CF₃COOH) to obtain the target product B11c with a yield of 70%.

MS (ESI): m/z 637.3 [M+H]⁺

¹H NMR (600 MHz, DMSO-d6) δ 8.22 (d, J=8.5 Hz, 1H), 7.93 (d, J=8.8 Hz, 2H), 7.59 (d, J=8.3 Hz, 2H), 7.31-7.24 (m, 2H), 7.16 (t, J=8.9 Hz, 2H), 7.04 (s, 2H), 4.20-4.12 (m, 2H), 3.98 (q, J=7.0 Hz, 4H), 3.71 (h, J=7.6, 7.1 Hz, 1H), 3.12 (dd, J=30.4, 11.9 Hz, 2H), 2.82 (q, J=10.2, 7.3 Hz, 1H), 2.72 (p, J=10.0 Hz, 1H), 2.18-2.11 (m, 1H), 1.95-1.76 (m, 4H), 1.69-1.53 (m, 3H), 1.16 (t, J=7.0 Hz, 6H).

### Example 12

### 1-((2,6-diethoxy-4′-fluoro-[1,1′-biphenyl]-4-yl)methyl)-4-(1-((4-(trifluoromethoxy)phenyl)sulfonyl)piperidin-4-yl)piperazine, trifluoroacetate

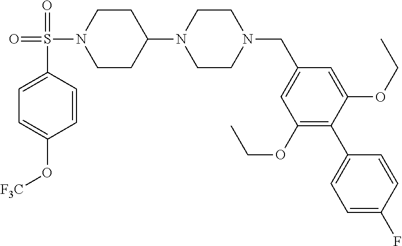 [SMILES: CCOc1cc(CN2CCN(C3CCN(S(=O)(=O)c4ccc(OC(F)(F)F)cc4)CC3)CC2)cc(OCC)c1-c1ccc(F)cc1]

The synthesis steps were the same as those in Example 11, except that tert-butyl 2-amino-7-azaspiro[3.5]nonane-7-carboxylate (CAS: 1239319-82-4) was replaced by tert-butyl 4-(piperidin-4-yl)piperazine-1-carboxylate (CAS: 205059-24-1).

MS (ESI): m/z 666.3 [M+H]⁺.

¹H NMR (500 MHz, DMSO-d6) δ 7.96-7.87 (m, 2H), 7.64 (d, J=8.4 Hz, 2H), 7.28 (dd, J=8.6, 5.8 Hz, 2H), 7.16 (t, J=8.9 Hz, 2H), 6.81 (s, 2H), 4.07 (s, 2H), 3.97 (q, J=6.9 Hz, 4H), 3.77 (d, J=11.6 Hz, 2H), 3.41-2.90 (m, 9H), 2.36-2.24 (m, 2H), 2.06-1.96 (m, 2H), 1.69-1.54 (m, 2H), 1.16 (t, J=7.0 Hz, 6H).

### Example 13

### 1-(1-((2,6-diethoxy-4′-fluoro-[1,1′-biphenyl]-4-yl)methyl)piperidin-4-yl)-4-((4-(trifluoromethoxy)phenyl))sulfonyl)piperazine, trifluoroacetate

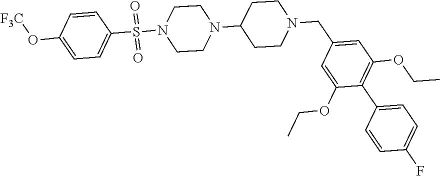 [SMILES: CCOc1cc(CN2CCC(N3CCN(S(=O)(=O)c4ccc(OC(F)(F)F)cc4)CC3)CC2)cc(OCC)c1-c1ccc(F)cc1]

The synthesis steps were the same as those in Example 11, except that tert-butyl 2-amino-7-azaspiro[3.5]nonane-7-carboxylate (CAS: 1239319-82-4) was replaced by tert-butyl 4-piperazinetetrahydro-1(2H)-pyridine carboxylate (CAS: 177276-41-4).

MS (ESI): m/z 666.3 [M+H]⁺.

¹H NMR (500 MHz, DMSO-d6) δ 7.91 (d, J=8.5 Hz, 2H), 7.67 (d, J=8.4 Hz, 2H), 7.28 (dd, J=8.7, 5.8 Hz, 2H), 7.17 (t, J=8.9 Hz, 2H), 6.86 (s, 2H), 4.26 (s, 2H), 3.97 (q, J=7.0 Hz, 4H), 3.86-3.37 (m, 6H), 3.34-3.06 (m, 4H), 3.06-2.85 (m, 2H), 2.18 (d, J=13.2 Hz, 2H), 1.88 (d, J=13.2 Hz, 2H), 1.16 (t, J=6.9 Hz, 6H).

### Example 14

### 1-((2,6-diethoxy-4′-fluoro-[1,1′-biphenyl]-4-yl)methyl))-4-(1-((4-(trifluoromethoxy)phenyl)sulfonyl)azetidin-3-yl)piperidine, trifluoroacetate

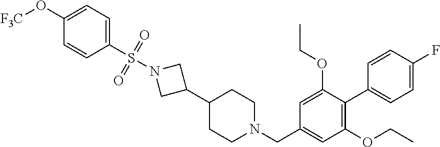 [SMILES: CCOc1cc(CN2CCC(C3CN(S(=O)(=O)c4ccc(OC(F)(F)F)cc4)C3)CC2)cc(OCC)c1-c1ccc(F)cc1]

The synthesis steps were the same as those in Example 11, except that tert-butyl 2-amino-7-azaspiro[3.5]nonane-7-carboxylate (CAS: 1239319-82-4) was replaced by tert-butyl 4-(azetidin-3-yl)piperidine-1-carboxylate (CAS: 1314703-47-3).

MS (ESI): m/z 636.2 [M+H]⁺.

¹H NMR (500 MHz, DMSO-d6) δ 8.06 (s, 1H), 7.97 (d, J=8.8 Hz, 2H), 7.83 (dd, J=8.6, 6.4 Hz, 1H), 7.68 (dt, J=7.7, 1.1 Hz, 2H), 7.26 (dd, J=8.7, 5.9 Hz, 2H), 7.19-7.08 (m, 3H), 6.58 (s, 2H), 3.91 (q, J=7.0 Hz, 4H), 3.80 (t, J=8.4 Hz, 2H), 3.41 (dd, J=8.5, 6.4 Hz, 2H), 3.38-3.34 (m, 2H), 2.74 (d, J=11.0 Hz, 2H), 2.22 (q, J=7.4 Hz, 1H), 1.71 (s, 2H), 1.41-1.31 (m, 2H), 1.14 (t, J=6.9 Hz, 6H), 0.94-0.78 (m, 3H).

### Example 15

### 4-((4-(4-((2,6-diethoxy-4′-fluoro-[1,1′-biphenyl]-4-yl)methyl) piperazin-1-yl)piperidin-1-yl)sulfonyl)benzoic acid, trifluoroacetate

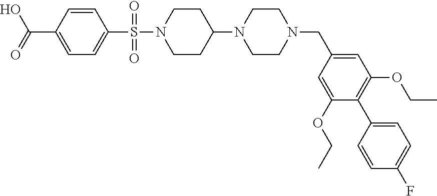 [SMILES: CCOc1cc(CN2CCN(C3CCN(S(=O)(=O)c4ccc(C(=O)O)cc4)CC3)CC2)cc(OCC)c1-c1ccc(F)cc1]

The preparation method was the same as that in Example 12, except that 4-(trifluoromethoxy)benzenesulfonyl chloride (CAS: 94108-56-2) was replaced by 4-(chlorosulfonyl)benzoic acid (CAS: 10130-89-9).

MS (ESI): m/z 626.3 [M+H]⁺.

¹H NMR (500 MHz, DMSO-d6) δ 7.67 (d, J=7.9 Hz, 2H), 7.35 (d, J=7.9 Hz, 2H), 7.32-7.24 (m, 2H), 7.17 (t, J=8.9 Hz, 2H), 6.81 (s, 2H), 4.60 (d, J=26.2 Hz, 1H), 4.23-4.01 (m, 2H), 3.98 (q, J=6.9 Hz, 4H), 3.44 (q, J=7.0 Hz, 4H), 3.20-2.89 (m, 4H), 2.79 (s, 2H), 2.14-1.82 (m, 2H), 1.56 (s, 2H), 1.17 (t, J=7.0 Hz, 6H), 1.05 (t, J=7.0 Hz, 2H).

### Example 16

### 1-((2,6-diethoxy-4′-fluoro-[1,1′-biphenyl]-4-yl)methyl)-4-((4-(trifluoromethoxy)phenyl)sulfonyl)piperazine

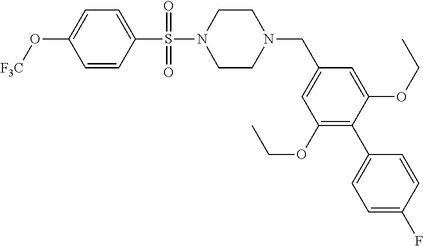 [SMILES: CCOc1cc(CN2CCN(S(=O)(=O)c3ccc(OC(F)(F)F)cc3)CC2)cc(OCC)c1-c1ccc(F)cc1]

The synthesis steps were the same as those in Example 12, except that tert-butyl 2-amino-7-azaspiro[3.5]nonane-7-carboxylate (CAS: 1239319-82-4) was replaced by 1-Boc-piperazine (CAS: 57260-71-6).

MS (ESI): m/z 583.2 [M+H]⁺.

¹H NMR (500 MHz, DMSO-d6) δ 7.92 (td, J=5.8, 2.0 Hz, 2H), 7.69 (ddd, J=10.9, 6.3, 3.9 Hz, 2H), 7.27 (ddd, J=8.5, 5.4, 1.9 Hz, 2H), 7.17 (td, J=8.9, 2.0 Hz, 2H), 6.83-6.78 (m, 2H), 4.31 (s, 2H), 3.96 (qd, J=6.9, 2.5 Hz, 4H), 3.55 (s, 8H), 1.16 (t, J=7.0 Hz, 6H).

### Example 17

### 4-((4-((2,6-diethoxy-4′-fluoro-[1,1′-biphenyl]-4-yl)methyl)piperazin-1-yl)sulfonyl)benzoic acid

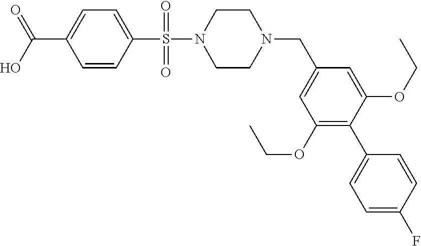 [SMILES: CCOc1cc(CN2CCN(S(=O)(=O)c3ccc(C(=O)O)cc3)CC2)cc(OCC)c1-c1ccc(F)cc1]

The preparation method was the same as that in Example 16, except that 4-(trifluoromethoxy)benzenesulfonyl chloride (CAS: 94108-56-2) was replaced by 4-(chlorosulfonyl)benzoic acid (CAS: 10130-89-9).

MS (ESI): m/z 543.2 [M+H]⁺.

¹H NMR (500 MHz, DMSO-d6) δ 8.20 (d, J=8.5 Hz, 2H), 7.89 (d, J=8.5 Hz, 2H), 7.26 (dd, J=8.7, 5.8 Hz, 2H), 7.20-7.12 (m, 2H), 6.79 (s, 2H), 4.25 (s, 2H), 3.95 (q, J=7.0 Hz, 4H), 3.79-3.40 (m, 8H), 1.15 (t, J=6.9 Hz, 6H).

### Example 18

### 4-(7-((2,6-diethoxy-4′-fluoro-[1,1′-biphenyl]-4-yl)methyl)-1-oxo-2,7-diazaspiro[3.5]nonan-2-yl)benzoic acid, trifluoroacetate

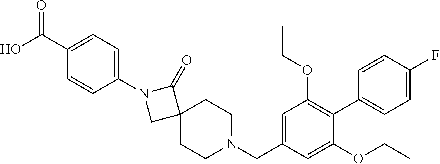 [SMILES: CCOc1cc(CN2CCC3(CC2)CN(c2ccc(C(=O)O)cc2)C3=O)cc(OCC)c1-c1ccc(F)cc1]

The synthesis steps were the same as those in Example 3, except that 3,9-diazaspiro[5.5]-2-undecanone (CAS: 867006-20-0) was replaced by 2,7-diazaspiro[3.5]nonan-1-one (CAS: 1147422-92-1).

MS (ESI): m/z 533.2 [M+H]⁺.

¹H NMR (500 MHz, DMSO-d6) δ 7.99 (dd, J=8.7, 3.0 Hz, 2H), 7.44 (d, J=8.7 Hz, 2H, 7.34-7.29 (m, 2H), 7.23-7.17 (m, 2H), 6.99 (s, 1H), 6.88 (s, 1H), 4.38 (dd, J=26.2, 5.2 Hz, 2H), 4.02 (q, J=7.0 Hz, 4H), 3.80 (s, 1H), 3.68 (s, 1H), 3.56-3.47 (m, 2H), 3.33-3.24 (m, 1H) 3.19-2.09 (m, 1H), 2.42 (d, J=14.0 Hz, 1H), 2.24 (d, J=14.1 Hz, 1H), 2.21-2.04 (m, 2H), 1.21 (t, J=7.0, 6H).

### Example 19

### 4-(7-(2-cyclopropyl-5-ethoxy-4-methylbenzyl)-2,7-diazaspiro[3,5]nonan-2-yl)benzoic acid, trifluoroacetate

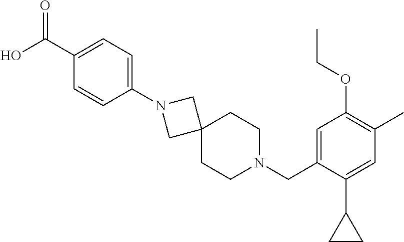 [SMILES: CCOc1cc(CN2CCC3(CC2)CN(c2ccc(C(=O)O)cc2)C3)c(C2CC2)cc1C]

The preparation method was the same as that in Example 10, except that the intermediate A1c was replaced by the intermediate A8e.

MS (ESI): m/z 435.3 [M+H]⁺.

¹H NMR (500 MHz, DMSO-d6) δ 9.41 (s, 1H), 7.76 (d, J=8.6 Hz, 2H), 7.07 (s, 1H), 6.83 (s, 1H), 6.41 (d, J=8.6 Hz, 2H), 4.44 (d, J=4.8 Hz, 2H), 4.03 (q, J=6.9 Hz, 2H), 3.79 (s, 2H), 3.68 (s, 2H), 3.40-3.33 (m, 2H), 3.19-3.09 (m, 2H), 2.19-2.12 (m, 2H), 2.12 (s, 3H), 2.09-2.05 (m, 1H), 1.99-1.91 (m, 2H), 1.35 (t, J=6.9 Hz, 3H), 0.97-0.91 (m, 2H), 0.65-0.60 (m, 2H).

### Example 20

### 4-(7-((4-ethoxy-2′,3′,4′-trifluoro-5-methyl-[1,1′-biphenyl]-2-yl)methyl)-2,7-diazaspiro[3,5]nonan-2-yl)benzoic acid, trifluoroacetate

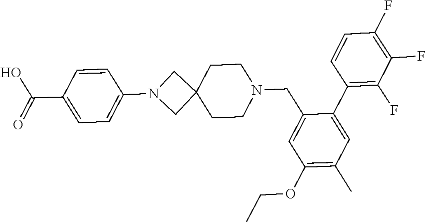 [SMILES: CCOc1cc(CN2CCC3(CC2)CN(c2ccc(C(=O)O)cc2)C3)c(-c2ccc(F)c(F)c2F)cc1C]

### Step 1: Preparation of Intermediate 2′-(chloromethyl)-4′-ethoxy-2,3,4-trifluoro-5′-methyl-1,1′-biphenyl (A21a)

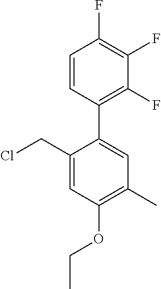 [SMILES: CCOc1cc(CCl)c(-c2ccc(F)c(F)c2F)cc1C]

The synthesis steps were the same as those for synthesizing intermediate A8e, except that cyclopropylboronic acid (CAS: 411235-57-9) was replaced by 2,3,4-trifluorophenylboronic acid (CAS: 226396-32-3).

MS (ESI): m/z 315.1 [M+H]⁺.

¹H NMR (500 MHz, Chloroform-d) δ 7.85 (s, 1H), 7.67 (d, J=8.0 Hz, 1H), 7.39 (d, J=8.0 Hz, 1H), 7.17-7.03 (m, 2H), 4.47 (s, 2H). 1H NMR (500 MHz, Chloroform-d) δ 7.08 (s, 1H), 7.07-7.00 (m, 3H), 4.51 (s, 2H), 4.15 (q, J=7.0 Hz, 2H), 2.27 (s, 3H), 1.49 (t, J=7.0 Hz, 3H).

### Step 2: Synthesis of the Final Product 4-(7-((4-ethoxy-2′,3′,4′-trifluoro-5-methyl-[1,1′-biphenyl]-2-yl)methyl)-2,7-diazaspiro[3,5]nonan-2-yl)benzoic acid

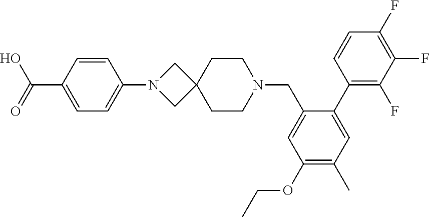 [SMILES: CCOc1cc(CN2CCC3(CC2)CN(c2ccc(C(=O)O)cc2)C3)c(-c2ccc(F)c(F)c2F)cc1C]

The synthesis steps are the same as those in Example 20, except that the intermediate A8e was replaced by the intermediate A21a.

MS (ESI): m/z 525.2 [M+H]⁺.

### Example 21

### 4-(2-(3-ethoxy-4-fluorobenzyl)-7-oxy-2,6-diazaspiro[3.4]octan-6-yl)benzoic acid, trifluoroacetate

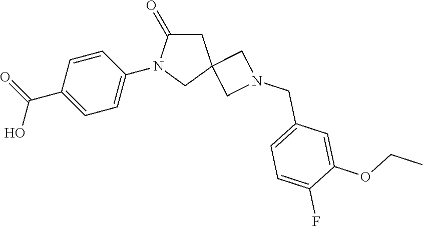 [SMILES: CCOc1cc(CN2CC3(CC(=O)N(c4ccc(C(=O)O)cc4)C3)C2)ccc1F]

### Step 1: Synthesis of Intermediate methyl 4-(2-(3-ethoxy-4-fluorobenzyl)-7-oxy-2,6-diazaspiro[3.4]octan-6-yl)benzoate (A22a)

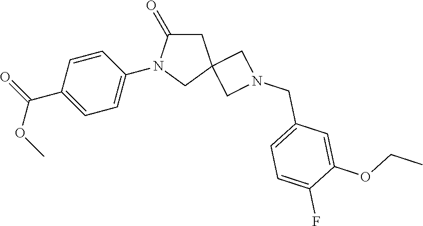 [SMILES: CCOc1cc(CN2CC3(CC(=O)N(c4ccc(C(=O)OC)cc4)C3)C2)ccc1F]

Intermediate A8b: methyl 4-(7-oxo-2,6-diazaspiro[3.4]octan-6-yl)benzoate (1 eq) and 3-ethoxy-4-fluorobenzaldehyde (1.1 eq) were dissolved in ultra-dry 1,2-dichloroethane and stirred at room temperature for 0.5 h. Sodium triacetoxyborohydride (2 eq) was added and stirred at room temperature overnight. After the reaction was complete as confirmed by TLC, the reaction solution was quenched with aqueous ammonium chloride solution, extracted three times with ethyl acetate, the organic layers were combined and dried over anhydrous magnesium sulfate, and the resultant was separated and purified by Flash column to obtain intermediate A22a.

MS (ESI): m/z 423.1 [M+H]⁺.

### Step 2: Preparation of the Final Product 4-(2-(3-ethoxy-4-fluorobenzyl)-7-oxy-2,6-diazaspiro[3.4]octan-6-yl)benzoic acid

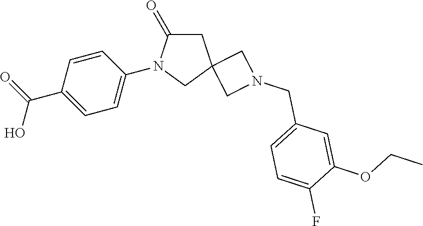 [SMILES: CCOc1cc(CN2CC3(CC(=O)N(c4ccc(C(=O)O)cc4)C3)C2)ccc1F]

The intermediate methyl 4-(2-(3-ethoxy-4-fluorobenzyl)-7-oxy-2,6-diazaspiro[3.4]octan-6-yl)benzate (1 eq) was dissolved in a mixed solvent of methanol, tetrahydrofuran and water (volume ratio=3:3:1), and lithium hydroxide monohydrate (2 eq) was added. The reaction was carried out at 50° C. for 12 hours. After the reaction was complete as confirmed by TLC, the reaction solution was concentrated by evaporation, and purified by semi-preparative liquid phase to obtain the final product with a yield of 70%.

MS (ESI): m/z 399.2 [M+H]⁺

### Example 22

### 4-(2-(3-ethoxy-4-methylbenzyl)-7-oxy-2,6-diazaspiro[3.4]octan-6-yl)benzoic acid, trifluoroacetate

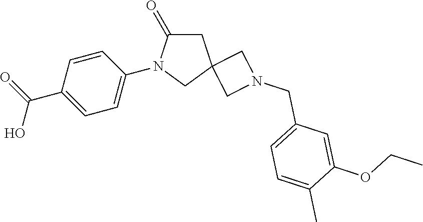 [SMILES: CCOc1cc(CN2CC3(CC(=O)N(c4ccc(C(=O)O)cc4)C3)C2)ccc1C]

The synthesis steps were the same as those in Example 21, except that 3-ethoxy-4-fluorobenzaldehyde was replaced by 3-ethoxy-4-methylbenzaldehyde.

MS (ESI): m/z 395.2 [M+H]⁺.

### Example 23

### 4-(2-(2-cyclopropyl-5-(trifluoromethyl)benzyl)-7-oxo-2,6-diazaspiro[3.4]octyl-6-yl)benzoic acid, trifluoroacetate

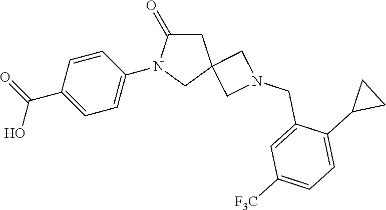 [SMILES: O=C(O)c1ccc(N2CC3(CC2=O)CN(Cc2cc(C(F)(F)F)ccc2C2CC2)C3)cc1]

### Step 1: Preparation of Intermediate 2-(chloromethyl)-1-cyclopropyl-4-(trifluoromethyl)benzene (A24a)

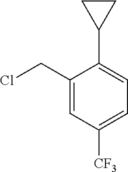 [SMILES: FC(F)(F)c1ccc(C2CC2)c(CCl)c1]

The synthesis steps were the same as those for synthesizing intermediate A8e, except that ethyl 2-bromo-5-ethoxy-4-methylbenzoate (CAS: 1350759-94-2) was replaced by ethyl 2-bromo-5-(trifluoromethyl)benzoate (CAS: 1214336-55-6).

¹H NMR (500 MHz, Chloroform-d) δ 7.60 (d, J=2.0 Hz, 1H), 7.50 (dd, J=8.1, 2.0 Hz, 1H), 7.12 (d, J=8.1 Hz, 1H), 4.82 (s, 2H), 2.13 (tt, J=8.5, 5.3 Hz, 1H), 1.13-1.05 (m, 2H), 0.80-0.73 (m, 2H).

### Step 2: Preparation of the Final Product 4-(2-(2-cyclopropyl-5-(trifluoromethyl)benzyl)-7-oxo-2,6-diazaspiro[3.4]octyl-6-yl)benzoic acid

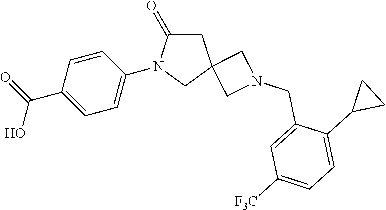 [SMILES: O=C(O)c1ccc(N2CC3(CC2=O)CN(Cc2cc(C(F)(F)F)ccc2C2CC2)C3)cc1]

The synthesis steps were the same as those in Example 8, except that the intermediate A8e was replaced by the intermediate A24a.

MS (ESI): m/z 445.2 [M+H]⁺.

### Example 24

### 4-(7-(3-ethoxy-4-fluorobenzyl)-2,7-diazaspiro[3.5]nonan-2-yl)benzoic acid, trifluoroacetate

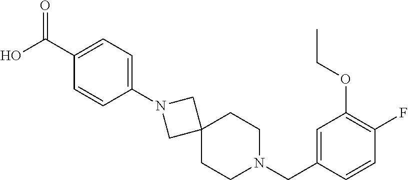 [SMILES: CCOc1cc(CN2CCC3(CC2)CN(c2ccc(C(=O)O)cc2)C3)ccc1F]

The preparation method was the same as that in Example 21, except that the intermediate A8b was replaced by the intermediate A10b.

MS (ESI): m/z 399.3 [M+H]⁺.

¹H NMR (500 MHz, Methanol-d4) δ 7.82 (d, J=8.7 Hz, 2H), 7.24 (d, J=7.9 Hz, 1H), 7.17 (dd, J=11.0, 8.3 Hz, 1H), 7.03 (dq, J=6.1, 1.9 Hz, 1H), 6.42 (d, J=8.7 Hz, 2H), 4.26 (s, 2H), 4.12 (q, J=7.0 Hz, 2H), 3.87-3.63 (m, 4H), 3.50-3.35 (m, 2H), 3.14-2.97 (m, 2H), 2.16 (s, 2H), 2.03 (s, 2H), 1.41 (t, J=7.0 Hz, 3H).

### Example 25

### 4-(7-(4-(diethylamino)-3-ethoxybenzyl)-2,7-diazaspiro[3.5]nonan-2-yl)benzoic acid, trifluoroacetate

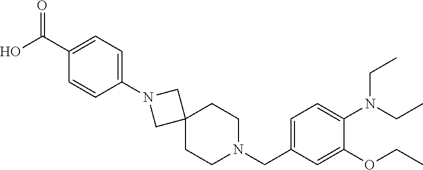 [SMILES: CCOc1cc(CN2CCC3(CC2)CN(c2ccc(C(=O)O)cc2)C3)ccc1N(CC)CC]

### Step 1: Preparation of Intermediate: methyl 4-(diethylamino)-3-ethoxybenzoate (A26a)

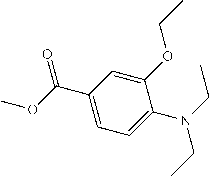 [SMILES: CCOc1cc(C(=O)OC)ccc1N(CC)CC]

Methyl 4-amino-3-hydroxybenzoate (1 eq) was dissolved in DMF, cesium carbonate (3.5 eq) was added, and then iodoethane 4 eq was added under stirring. The mixture was reacted at 70° C. overnight. After the reaction was complete as confirmed by TLC, the reaction solution was extracted three times with ethyl acetate and water system, and the organic layers were combined and washed three times with saturated sodium chloride solution. The organic layer was dried over anhydrous magnesium sulfate, filtered, and the filtrate was mixed with silica gel and separated by Flash column to obtain the target product A26a.

MS (ESI): m/z 252.2 [M+H]⁺.

¹H NMR (500 MHz, Chloroform-d) δ 7.57 (dd, J=8.3, 1.5 Hz, 1H), 7.48 (s, 1H), 6.83 (s, 1H), 4.11 (q, J=7.0 Hz, 2H), 3.86 (s, 3H), 3.29 (q, J=7.1 Hz, 4H), 1.46 (t, J=7.0 Hz, 3H), 1.11 (t, J=7.0 Hz, 6H).

### Step 2: Preparation of Intermediate (4-(diethylamino)-3-ethoxyphenyl)methanol (A26b)

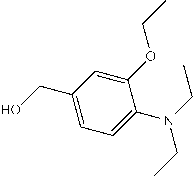 [SMILES: CCOc1cc(CO)ccc1N(CC)CC]

The intermediate A21a (1 eq) obtained in the previous step was dissolved in ultra-dry tetrahydrofuran, and 2.4 M lithium aluminum tetrahydride solution in tetrahydrofuran (1 eq) was added under stirring at room temperature, and the mixture was reacted at room temperature for 1 h. After the reaction was complete as confirmed by TLC, the reaction solution was quenched with 0.5 M sodium hydroxide aqueous solution, filtered on diatomite, and the filter cake was repeatedly rinsed with ethyl acetate. The filtrate was evaporated to dryness and then re-dissolved in ethyl acetate, mixed with silica gel, and separated by Flash column to obtain the target product A26b.

MS (ESI): m/z 224.2 [M+H]⁺.

¹H NMR (500 MHz, Chloroform-d) δ 6.92-6.83 (m, 3H), 4.61 (s, 2H), 4.09 (q, J=6.9 Hz, 2H), 3.16 (q, J=7.0 Hz, 4H), 1.45 (t, J=7.0 Hz, 3H), 1.04 (t, J=7.1 Hz, 6H).

### Step 3: Preparation of Intermediate 4-(chloromethyl)-2-ethoxy-N,N-diethylaniline (A26c)

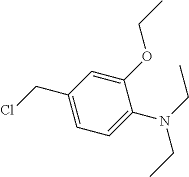 [SMILES: CCOc1cc(CCl)ccc1N(CC)CC]

The intermediate A26b obtained in the previous step was dissolved in dichloromethane, and an excess of thionyl chloride was added under stirring at room temperature, and the reaction was carried out at room temperature for 30 minutes. After the reaction was complete as confirmed by TLC, the reaction solution was evaporated off the solvent to obtain intermediate A26c.

MS (ESI): m/z 242.2 [M+H]⁺.

### Step 4: Preparation of Intermediate methyl 4-(7-(4-(diethylamino)-3-ethoxybenzyl)-2,7-diazaspiro[3.5]nonan-2-yl)benzoate (A26d)

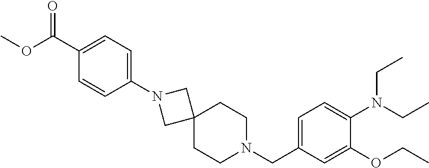 [SMILES: CCOc1cc(CN2CCC3(CC2)CN(c2ccc(C(=O)OC)cc2)C3)ccc1N(CC)CC]

Intermediate A10b (1eq), intermediate A26c (1.5eq), and potassium carbonate (2eq) were dissolved in DMF and stirred at room temperature overnight. After the reaction was complete as confirmed by TLC, the reaction solution was extracted 3 times with ethyl acetate and water system. The organic layers were combined and washed 3 times with saturated sodium chloride solution. The organic layer was dried over anhydrous magnesium sulfate, filtered, and the filtrate was evaporated to dryness and separated by a preparative TLC plate to obtain the target product A26d.

MS (ESI): m/z 466.3 [M+H]⁺.

¹H NMR (500 MHz, Chloroform-d) δ 7.91 (d, J=8.8 Hz, 2H), 6.89 (d, J=8.0 Hz, 1H), 6.88 (s, 1H) 6.81 (d, J=8.0 Hz, 1H), 6.38 (d, J=8.8 Hz, 2H), 4.12 (q, J=7.0 Hz, 2H), 3.88 (s, 3H), 3.69 (s, 4H), 3.46 (s, 2H), 3.19 (q, J=7.0 Hz, 4H), 2.42 (br, 4H), 1.88 (br, 4H), 1.48 (t, J=7.0 Hz, 3H), 1.08 (t, J=7.1 Hz, 6H).

### Step 5: Preparation of the Final Product 4-(7-(4-(diethylamino)-3-ethoxybenzyl)-2,7-diazaspiro[3.5]nonan-2-yl)benzoic acid

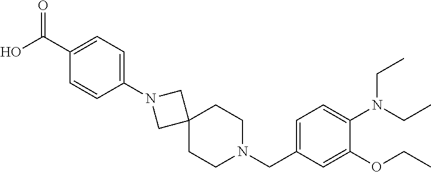 [SMILES: CCOc1cc(CN2CCC3(CC2)CN(c2ccc(C(=O)O)cc2)C3)ccc1N(CC)CC]

The intermediate A26d: methyl 4-(7-(4-(diethylamino)-3-ethoxybenzyl)-2,7-diazaspiro[3.5]nonan-2-yl)benzoate (1 eq) was dissolved in a mixed solvent of methanol, tetrahydrofuran and water (volume ratio=3:3:1), and lithium hydroxide monohydrate (2 eq) was added. The reaction was carried out at 50° C. for 12 hours. After the reaction was complete as confirmed by TLC, the reaction solution was concentrated by evaporation, and purified by semi-preparative liquid phase to obtain the final product with a yield of 70%.

MS (ESI): m/z 452.3 [M+H]⁺.

¹H NMR (500 MHz, Methanol-d4) δ 7.83 (d, J=8.8 Hz, 2H), 7.68-7.60 (m, 1H), 7.45 (d, J=7.1 Hz, 1H), 7.30 (d, J=8.1 Hz, 1H), 6.44 (d, J=8.8 Hz, 2H), 4.36 (s, 2H), 4.31 (q, J=7.0 Hz, 2H), 3.78 (br, 4H), 3.62 (br, 4H), 2.13 (br, 4H), 1.49 (t, J=7.0 Hz, 3H), 1.08 (t, J=7.2 Hz, 6H).

### Example 26

### 4-(2-(3-ethoxy-4-hydroxybenzyl)-7-oxo-2,6-diazaspiro[3.4]octyl-6-yl)benzoic acid, trifluoroacetate

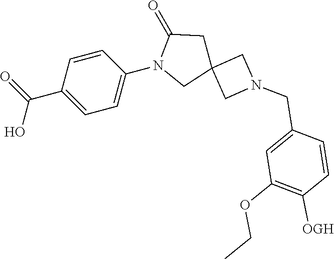

The synthesis steps were the same as those in Example 21, except that 3-ethoxy-4-fluorobenzaldehyde was replaced by 3-ethoxy-4-hydroxybenzaldehyde.

MS (ESI): m/z 397.2 [M+H]⁺.

¹H NMR (500 MHz, DMSO-d6) δ 7.89 (d, J=8.5 Hz, 2H), 7.59 (d, J=8.6 Hz, 2H), 6.80 (d, J=1.7 Hz, 1H), 6.72 (d, J=8.0 Hz, 1H), 6.65 (dd, J=8.0, 1.7 Hz, 1H), 4.01 (s, 2H), 4.00 (q, J=6.8 Hz, 2H), 3.44 (s, 2H), 3.21 (d, J=7.1 Hz, 2H), 3.16 (d, J=7.0 Hz, 2H), 2.76 (s, 2H), 1.32 (t, J=7.0 Hz, 3H).

### Example 27

### 4-(7-((2,6-diethoxy-4′-fluoro-[1,1′-biphenyl]-4-yl)methyl)-2,7-diazaspiro[3.5]nonan-2-yl)-3-fluorobenzoic acid, trifluoroacetate

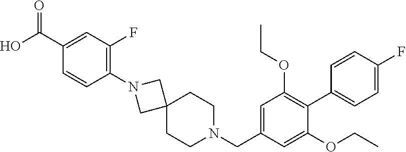 [SMILES: CCOc1cc(CN2CCC3(CC2)CN(c2ccc(C(=O)O)cc2F)C3)cc(OCC)c1-c1ccc(F)cc1]

The synthesis steps were the same as those in Example 10, except that methyl p-bromobenzoate was replaced by methyl 3-fluoro-4-bromobenzoate.

MS (ESI): m/z 537.3 [M+H]⁺.

¹H NMR (500 MHz, DMSO-d6) δ 9.94 (s, 1H), 7.62 (d, J=8.2 Hz, 1H), 7.50 (d, J=13.4 Hz, 1H), 7.34-7.26 (m, 2H), 7.18 (t, J=8.7 Hz, 2H), 6.87 (s, 2H), 6.53 (t, J=8.7 Hz, 1H), 4.29 (s, 2H), 3.99 (q, J=6.8 Hz, 4H), 3.95 (s, 2H), 3.81 (s, 2H), 3.35 (d, J=11.7 Hz, 2H), 3.04 (q, J=10.4, 9.9 Hz, 2H), 2.19 (d, J=13.7 Hz, 2H), 1.93 (t, J=12.1 Hz, 2H), 1.18 (t, J=6.8 Hz, 6H).

### Example 28

### 4-(7-((2,6-diethoxy-4′-fluoro-[1,1′-biphenyl]-4-yl)methyl)-2,7-diazaspiro[3.5]nonan-2-yl)-2-methoxybenzoic acid, trifluoroacetate

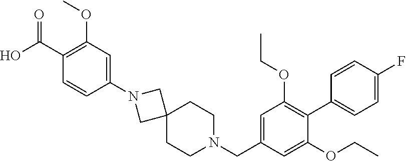 [SMILES: CCOc1cc(CN2CCC3(CC2)CN(c2ccc(C(=O)O)c(OC)c2)C3)cc(OCC)c1-c1ccc(F)cc1]

The synthesis steps were the same as those in Example 10, except that methyl p-bromobenzoate was replaced by methyl 4-bromo-2-methoxybenzoate.

MS (ESI): m/z 549.3 [M+H]⁺.

¹H NMR (500 MHz, DMSO-d6) δ 7.89 (d, J=8.5 Hz, 2H), 7.59 (d, J=8.6 Hz, 2H), 6.80 (d, J=1.7 Hz, 1H), 6.72 (d, J=8.0 Hz, 1H), 6.65 (dd, J=8.0, 1.7 Hz, 1H), 4.01 (s, 2H), 4.00 (q, J=6.8 Hz, 2H), 3.44 (s, 2H), 3.21 (d, J=7.1 Hz, 2H), 3.16 (d, J=7.0 Hz, 2H), 2.76 (s, 2H), 1.32 (t, J=7.0 Hz, 3H).

### Example 29

### 5-(7-(2-cyclopropyl-5-ethoxy-4-methylbenzyl)-2,7-diazaspiro[3.5]nonan-2-yl) picolinic acid (29)

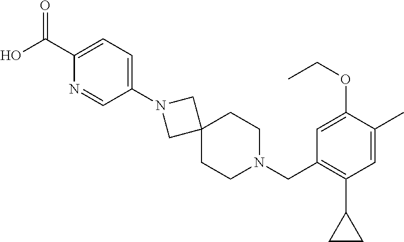 [SMILES: CCOc1cc(CN2CCC3(CC2)CN(c2ccc(C(=O)O)nc2)C3)c(C2CC2)cc1C]

### Step 1: Preparation of tert-butyl 7-(2-cyclopropyl-5-ethoxy-4-methylbenzyl)-2,7-diaza[3.5]nonane-2-carboxylate (29a)

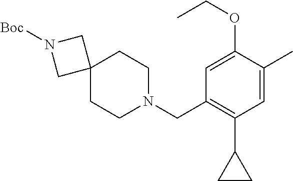 [SMILES: CCOc1cc(CN2CCC3(CC2)CN(C(=O)OC(C)(C)C)C3)c(C2CC2)cc1C]

A8e (1 eq), 2-tert-butyloxycarbonyl-2,7-diazaspiro[3.5]nonane (1 eq) and potassium carbonate (2 eq) were dissolved in acetonitrile and reacted overnight at room temperature. After the reaction was complete as confirmed by TLC, the reaction solution was mixed with silica gel and subjected to column chromatography to obtain intermediate 29a with a yield of 79%. ¹H NMR (500 MHz, Chloroform-d) δ 7.12 (s, 1H), 6.79 (s, 1H), 4.06 (q, J=6.9 Hz, 2H), 3.92 (s, 2H), 3.64 (s, 4H), 2.83-2.47 (m, 4H), 2.17 (s, 3H), 2.00-1.91 (m, 4H), 1.90-1.83 (m, 1H), 1.44 (s, 9H), 1.40 (t, J=6.9 Hz, 3H), 0.95-0.81 (m, 2H), 0.64-0.49 (m, 2H).

### Step 2: Preparation of 7-(2-cyclopropyl-5-ethoxy-4-methylbenzyl)-2,7-diaza[3.5]nonane hydrochloride (29b)

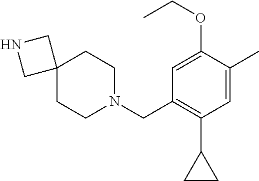 [SMILES: CCOc1cc(CN2CCC3(CC2)CNC3)c(C2CC2)cc1C]

29a was dissolved in dichloromethane, and an excess of hydrogen chloride-dioxane solution (4N) was added at room temperature. The mixture was stirred at room temperature for 2 hours. After the reaction was complete as confirmed by TLC, the reaction solution was concentrated under reduced pressure and used directly in the next step without purification.

### Step 3: Preparation of methyl 5-(7-(2-cyclopropyl-5-ethoxy-4-methylbenzyl)-2,7-diazaspiro[3.5]nonan-2-yl)picolinate (29c)

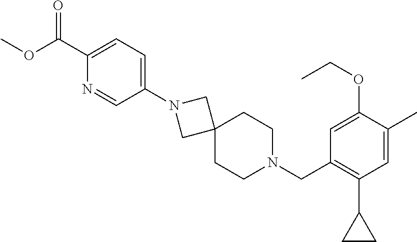

Methyl 5-fluoropicolinate (1.05 eq), 29b (1 eq) and potassium carbonate (2 eq) were dissolved in DMF and reacted at 90° C. overnight. After the reaction was complete as confirmed by TLC, the reaction solution was added with ice water, and extracted three times with ethyl acetate, the organic phases were combined and washed three times with saturated brine. The organic phases were dried, mixed with silica gel and subjected to column chromatography to obtain intermediate 29c with a yield of 68%. ¹H NMR (500 MHz, Chloroform-d) δ 8.76 (d, J=2.1 Hz, 1H), 7.98 (dd, J=8.8, 2.3 Hz, 1H), 6.87 (s, 1H), 6.78 (s, 1H), 6.19 (d, J=8.8 Hz, 1H), 4.03 (q, J=7.0 Hz, 2H), 3.86 (s, 3H), 3.82 (s, 4H), 3.60 (s, 2H), 2.44 (s, 4H), 2.17 (s, 3H), 2.02-1.95 (m, 1H), 1.84 (t, J=5.5 Hz, 4H), 1.40 (t, J=6.9 Hz, 3H), 0.89-0.80 (m, 2H), 0.62-0.53 (m, 2H).

### Step 4: Preparation of 5-(7-(2-cyclopropyl-5-ethoxy-4-methylbenzyl)-2,7-diazaspiro[3.5]nonan-2-yl)picolinic acid (29)

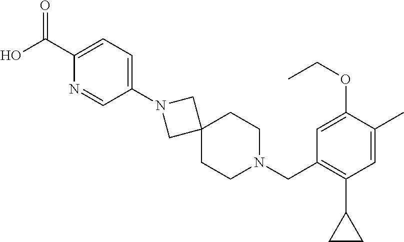 [SMILES: CCOc1cc(CN2CCC3(CC2)CN(c2ccc(C(=O)O)nc2)C3)c(C2CC2)cc1C]

29c was dissolved in a mixed solution of 1,4-dioxane and water, and lithium hydroxide monohydrate was added. The mixture was reacted at 60° C. overnight. After the reaction was complete as confirmed by TLC, the reaction solution was concentrated, adjusted to neutral pH with dilute hydrochloric acid, and the final product 29 was purified by semi-preparative liquid phase. ¹H NMR (500 MHz, Chloroform-d) δ 7.98 (d, J=8.5 Hz, 1H), 7.69 (s, 1H), 6.87 (s, 1H), 6.78 (s, 1H), 6.75-6.67 (m, 1H), 4.01 (q, J=7.0 Hz, 2H), 3.74 (s, 4H), 3.64 (s, 2H), 2.53-2.38 (m, 4H), 2.17 (s, 3H), 2.04-1.93 (m, 1H), 1.87 (t, J=5.4 Hz, 4H), 1.39 (t, J=6.9 Hz, 3H), 0.86 (td, J=8.2, 2.9 Hz, 2H), 0.57 (dd, J=5.6, 1.6 Hz, 2H).

### Example 30

### 6-(7-(2-cyclopropyl-5-ethoxy-4-methylbenzyl)-2,7-diazaspiro[3.5]nonan-2-yl) nicotinic acid (30)

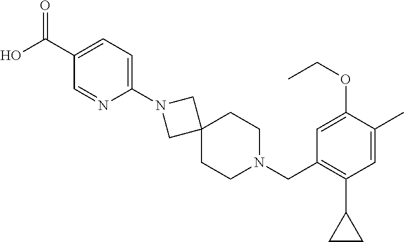 [SMILES: CCOc1cc(CN2CCC3(CC2)CN(c2ccc(C(=O)O)cn2)C3)c(C2CC2)cc1C]

### Preparation Method:

The preparation method was the same as that in Example 29, except that methyl 5-fluoropicolinate was replaced by methyl 6-fluoronicotinate.

¹H NMR (500 MHz, Chloroform-d) δ 8.78 (s, 1H), 8.03-7.95 (m, 1H), 6.91 (s, 1H), 6.75 (s, 1H), 6.10 (d, J=8.8 Hz, 1H), 3.95 (q, J=7.0 Hz, 2H), 3.81-3.71 (m, 6H), 2.71-2.45 (m, 4H), 2.15 (s, 3H), 1.96-1.90 (m, 1H), 1.33 (t, J=6.9 Hz, 3H), 0.85 (d, J=8.2 Hz, 2H), 0.55 (d, J=5.2 Hz, 2H).

### Example 31

### 4-(2-((6-cyclopropyl-2,4′-difluoro-3-isopropoxy-[1,1′-biphenyl]-4-yl)methyl)-7-oxo-2,6-diaza[3.4]octyl-6-yl)benzoic acid (31)

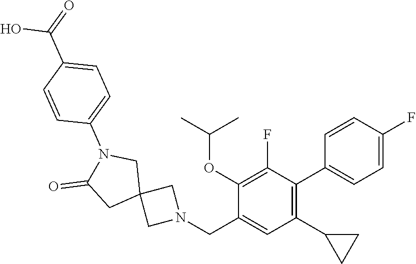 [SMILES: CC(C)Oc1c(CN2CC3(CC(=O)N(c4ccc(C(=O)O)cc4)C3)C2)cc(C2CC2)c(-c2ccc(F)cc2)c1F]

### Preparation Method:

### Step 1: Preparation of 1-bromo-3-(ethoxymethoxy)-2-fluorobenzene (31a)

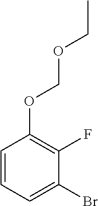

3-Bromo-2-fluorophenol (1 eq), chloromethyl ethyl ether (1.2 eq) and DIPEA (1.5 eq) were dissolved in tetrahydrofuran and reacted overnight at room temperature. After the reaction was complete as confirmed by TLC, the reaction solution was concentrated and redissolved in ethyl acetate and washed three times with saturated aqueous ammonium chloride solution. The organic phase was dried and concentrated to obtain intermediate 31a with a yield of 91%. ¹H NMR (500 MHz, Chloroform-d) δ 7.17 (ddd, J=8.6, 6.7, 1.9 Hz, 2H), 6.93 (td, J=8.2, 1.8 Hz, 1H), 5.26 (s, 2H), 3.77 (q, J=7.1 Hz, 2H), 1.23 (t, J=7.1 Hz, 4H).

### Step 2: Preparation of 3-(ethoxymethoxy)-2,4′-difluoro-1,1′-biphenyl (31b)

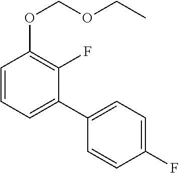 [SMILES: CCOCOc1cccc(-c2ccc(F)cc2)c1F]

31a (1 eq), p-fluorophenylboronic acid (1.5 eq), and potassium carbonate (3 eq) were dissolved in a mixed solvent of toluene and water (v:v=3:2). After nitrogen substitution and the addition of Pd₂(dba)₃ (0.05 eq), 2-dicyclohexylphosphino-2′,6′-dimethoxybiphenyl (0.1 eq), the reaction was carried out at 100° C. for 3 hours under nitrogen atmosphere. After the reaction was complete as confirmed by TLC, the reaction solution was cooled to room temperature, extracted with ethyl acetate, and the organic phases were combined, dried, and purified by column chromatography to obtain intermediate 31b with a yield of 81%. ¹H NMR (500 MHz, Chloroform-d) δ 7.50 (ddd, J=8.8, 5.4, 1.7 Hz, 2H), 7.21 (td, J=7.9, 1.7 Hz, 1H), 7.15-7.07 (m, 3H), 7.02 (ddd, J=7.9, 6.6, 1.7 Hz, 1H), 5.30 (s, 2H), 3.81 (q, J=7.1 Hz, 2H), 1.25 (t, J=7.1 Hz, 3H).

### Step 3: Preparation of 3-(ethoxymethoxy)-2,4′-difluoro-[1,1′-biphenyl]-4-carbaldehyde (31c)

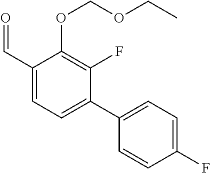 [SMILES: CCOCOc1c(C=O)ccc(-c2ccc(F)cc2)c1F]

31b (1 eq) was dissolved in ultra-dry tetrahydrofuran, stirred at −78° C. for 40 minutes, and then n-butyl lithium-n-hexane solution (1.05 eq) was added dropwise. Thereafter, the mixture was stirred at −78° C. for 1 hour, and then N,N-dimethylformamide solution (1.1 eq) was slowly added dropwise, and the mixture was slowly raised to 0° C. and reacted for 2 hours. After the reaction was complete as confirmed by TLC, the reaction was quenched by adding an ammonium chloride aqueous solution to the reaction solution, followed by extracting with ethyl acetate. The organic phases were combined, dried, and the reaction solution was concentrated. Intermediate 31c was obtained by column chromatography purification with a yield of 86%. ¹H NMR (500 MHz, Chloroform-d) δ 10.41 (d, J=0.8 Hz, 1H), 7.68 (dd, J=8.2, 1.4 Hz, 1H), 7.54 (ddd, J=8.8, 5.3, 1.8 Hz, 2H), 7.28-7.21 (m, 2H), 7.16 (t, J=8.7 Hz, 2H), 5.35 (s, 2H), 3.86 (q, J=7.1 Hz, 2H), 1.24 (t, J=7.1 Hz, 3H).

### Step 4: Preparation of 2,4′-difluoro-3-hydroxy-[1,1′-biphenyl]-4-carboxaldehyde (31d)

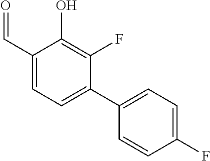 [SMILES: O=Cc1ccc(-c2ccc(F)cc2)c(F)c1O]

31c was dissolved in ethanol, excess concentrated hydrochloric acid was added, and the mixture was heated to 50° C. and reacted for 0.5 hours. The reaction mixture was then placed at room temperature for reaction for 1 hour. A white solid was precipitated from the reaction mixture. The reaction mixture was filtered and the filter cake was washed with water to obtain intermediate 31d. ¹H NMR (500 MHz, Chloroform-d) δ 11.11 (s, 1H), 9.94 (d, J=1.7 Hz, 1H), 7.57 (ddd, J=8.8, 5.3, 1.8 Hz, 2H), 7.42 (dd, J=8.2, 1.5 Hz, 1H), 7.17 (t, J=8.7 Hz, 2H), 7.05 (dd, J=8.1, 6.2 Hz, 1H).

### Step 5: Preparation of 6-bromo-2,4′-difluoro-3-hydroxy-[1,1′-biphenyl]-4-carbaldehyde (31e)

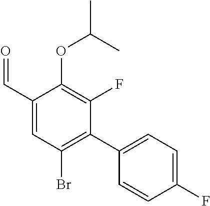 [SMILES: CC(C)Oc1c(C=O)cc(Br)c(-c2ccc(F)cc2)c1F]

31d (1 eq) was dissolved in DMF and 1,3-dibromo-1,3,5-triazine-2,4,6-trione (1.05 eq) was added, and reacted at room temperature under nitrogen protection for 6 hours. After the reaction was complete as confirmed by TLC, the reaction was quenched by adding water, and the reaction mixture was extracted with ethyl acetate. The organic phases were combined and washed with saturated brine three times, then dried, mixed with silica gel and subjected to column chromatography to obtain intermediate 31e with a yield of 80%. ¹H NMR (500 MHz, Chloroform-d) δ 11.11 (s, 1H), 9.94 (d, J=1.8 Hz, 1H), 7.58 (ddt, J=6.9, 5.2, 1.7 Hz, 1H), 7.43 (dd, J=8.1, 1.5 Hz, 1H), 7.22-7.13 (m, 2H), 7.05 (dd, J=8.1, 6.2 Hz, 1H).

### Step 6: Preparation of 6-bromo-2,4′-difluoro-3-isopropoxy-[1,1′-biphenyl]-4-carbaldehyde (31f)

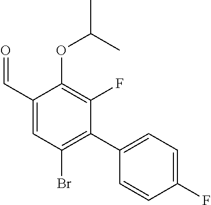 [SMILES: CC(C)Oc1c(C=O)cc(Br)c(-c2ccc(F)cc2)c1F]

31e (1 eq) was dissolved in DMF, and 2-iodopropane (1.2 eq) and potassium carbonate (1.5 eq) were added thereto and reacted at 60° C. overnight. After the reaction was complete as confirmed by TLC, the reaction was quenched by adding saturated brine, extracted with ethyl acetate, and the organic phases were combined and washed with water 3 times. Finally, the organic phase was dried, concentrated and mixed with silica gel and subjected to column chromatography to obtain intermediate 31f with a yield of 89%. ¹H NMR (500 MHz, Chloroform-d) δ 10.37 (s, 1H), 7.93 (d, J=1.8 Hz, 1H), 7.35-7.28 (m, 2H), 7.18 (td, J=8.7, 7.0 Hz, 2H), 4.73-4.56 (m, 1H), 1.40 (ddd, J=9.0, 6.1, 0.8 Hz, 6H).

### Step 7: Preparation of methyl 4-(2-((6-cyclopropyl-2,4′-difluoro-3-isopropoxy-[1,1′-biphenyl]-4-yl)methyl)-7-oxo-2,6-diaza[3.4]octyl-6-yl)benzoate (31g)

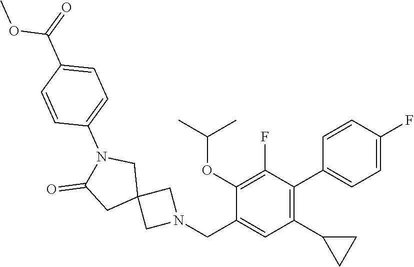 [SMILES: COC(=O)c1ccc(N2CC3(CC2=O)CN(Cc2cc(C4CC4)c(-c4ccc(F)cc4)c(F)c2OC(C)C)C3)cc1]

31f (1 eq), and A8b (1 eq) were dissolved in ultra-dry 1,2-dichloroethane and dehydrated with molecular sieves, followed by the addition of sodium acetate borohydride (3.5 eq), and the reaction was allowed to proceed overnight at room temperature, and the reaction was complete as confirmed by TLC. The reaction solution was directly mixed with silica gel and subjected to column chromatography to obtain intermediate 31g, with a yield of 81%. ¹H NMR (500 MHz, Chloroform-d) δ 8.04 (d, J=8.9 Hz, 2H), 7.74-7.66 (m, 2H), 7.33 (dd, J=8.5, 5.5 Hz, 2H), 7.14 (t, J=8.7 Hz, 2H), 6.72 (d, J=1.4 Hz, 1H), 4.47 (p, J=6.1 Hz, 1H), 4.12 (s, 2H), 3.91 (s, 3H), 3.89 (s, 2H), 3.70 (d, J=8.5 Hz, 2H), 3.56 (d, J=8.5 Hz, 2H), 2.87 (s, 2H), 1.60 (tt, J=8.4, 5.3 Hz, 1H), 1.33 (d, J=6.1 Hz, 6H), 0.83-0.74 (m, 2H), 0.71-0.62 (m, 2H).

### Step 8: Preparation of 4-(2-((6-cyclopropyl-2,4′-difluoro-3-isopropoxy-[1,1′-biphenyl]-4-yl)methyl)-7-oxo-2,6-diaza[3.4]octyl-6-yl)benzoic acid (31)

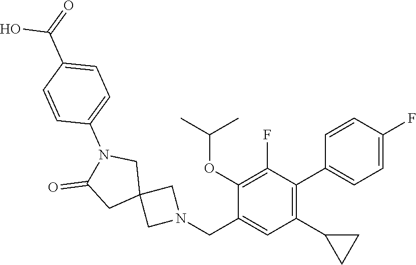 [SMILES: CC(C)Oc1c(CN2CC3(CC(=O)N(c4ccc(C(=O)O)cc4)C3)C2)cc(C2CC2)c(-c2ccc(F)cc2)c1F]

The synthesis method was the same as that in Example 29 except that 29c was replaced by 31g. ¹H NMR (600 MHz, DMSO-d₆) δ 7.89 (d, J=8.7 Hz, 2H), 7.57 (d, J=8.8 Hz, 2H), 7.43-7.37 (m, 2H), 7.29 (t, J=8.9 Hz, 2H), 6.73 (d, J=1.2 Hz, 1H), 4.32 (p, J=6.1 Hz, 1H), 4.02 (s, 2H), 3.60 (s, 2H), 3.29 (d, J=7.0 Hz, 2H), 3.26 (d, J=6.9 Hz, 2H), 2.78 (s, 2H), 1.55 (tt, J=8.4, 5.3 Hz, 1H), 1.26 (d, J=6.1 Hz, 6H), 0.75 (dd, J=8.4, 2.0 Hz, 2H), 0.60 (dd, J=5.3, 1.9 Hz, 2H).

### Example 32

### 4-(7-(2-cyclopropyl-5-isopropoxy-4-methylbenzyl)-2,7-diazaspiro[3.5]nonan-2-yl)benzoic acid (32)

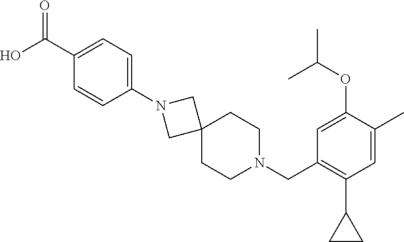 [SMILES: Cc1cc(C2CC2)c(CN2CCC3(CC2)CN(c2ccc(C(=O)O)cc2)C3)cc1OC(C)C]

The preparation method was the same as that in Example 19, except that iodoethane was replaced by 2-iodopropane. ¹H NMR (600 MHz, Methanol-d₄) δ 7.82 (d, J=8.7 Hz, 2H), 6.91 (s, 1H), 6.76 (s, 1H), 6.39 (d, J=8.8 Hz, 2H), 4.52 (p, J=6.1 Hz, 1H), 3.63 (s, 2H), 3.62 (s, 4H), 2.59-2.35 (m, 4H), 1.99 (tt, J=8.4, 5.4 Hz, 1H), 1.84 (t, J=5.5 Hz, 4H), 1.30 (d, J=6.0 Hz, 6H), 0.90-0.82 (m, 2H), 0.57-0.48 (m, 2H).

### Example 33

### 4-(7-((6-cyclopropyl-2,4′-difluoro-3-isopropoxy-[1,1′-biphenyl]-4-yl)methyl)-2,7-diaza[3.5]nonan-2-yl)-2-fluorobenzoic acid (33)

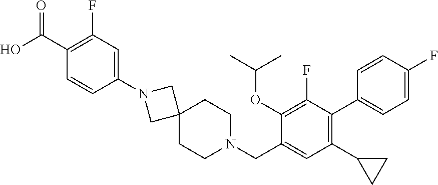 [SMILES: CC(C)Oc1c(CN2CCC3(CC2)CN(c2ccc(C(=O)O)c(F)c2)C3)cc(C2CC2)c(-c2ccc(F)cc2)c1F]

### Step 1: Preparation of tert-butyl 2-(2-fluoro-4-(methoxycarbonyl)phenyl)-2,7-diaza[3.5]nonane-7-carboxylate (33a)

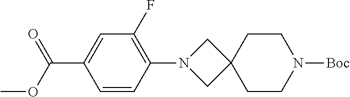 [SMILES: COC(=O)c1ccc(N2CC3(CCN(C(=O)OC(C)(C)C)CC3)C2)c(F)c1]

The preparation method was the same as that for A10a except that methyl p-bromobenzoate was replaced by methyl 4-bromo-3-fluorobenzoate. ¹H NMR (500 MHz, Chloroform-d) δ 7.68 (dd, J=8.4, 1.9 Hz, 1H), 7.59 (dd, J=13.4, 1.8 Hz, 1H), 6.36 (t, J=8.7 Hz, 1H), 3.86 (s, 3H), 3.84 (d, J=2.3 Hz, 4H), 3.46-3.35 (m, 4H), 1.79 (dd, J=6.7, 4.5 Hz, 4H), 1.47 (s, 9H).

### Step 2: Preparation of 4-(7-((6-cyclopropyl-2,4′-difluoro-3-isopropoxy-[1,1′-biphenyl]-4-yl)methyl)-2,7-diaza[3.5]nonan-2-yl)-2-fluorobenzoic acid (33)

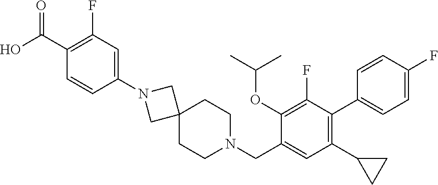 [SMILES: CC(C)Oc1c(CN2CCC3(CC2)CN(c2ccc(C(=O)O)c(F)c2)C3)cc(C2CC2)c(-c2ccc(F)cc2)c1F]

The preparation method was the same as that in Example 31 except that A8b was replaced by 33a. ¹H NMR (600 MHz, DMSO-d₆) δ 7.54 (dd, J=8.0, 1.7 Hz, 1H), 7.47 (dd, J=13.9, 1.7 Hz, 1H), 7.41 (dd, J=8.5, 5.6 Hz, 2H), 7.29 (t, J=8.9 Hz, 2H), 6.81 (s, 1H), 6.39 (t, J=8.7 Hz, 1H), 4.35 (p, J=6.1 Hz, 1H), 3.66 (s, 4H), 3.45 (s, 2H), 2.36 (s, 4H), 1.76 (t, J=5.6 Hz, 4H), 1.56 (td, J=8.4, 4.2 Hz, 1H), 1.25 (d, J=6.2 Hz, 6H), 0.75 (dt, J=8.6, 3.1 Hz, 2H), 0.59-0.51 (m, 2H).

### Example 34

### 4-(7-(2-cyclopropyl-5-ethoxy-4-methylbenzyl)-2,7-diazaspiro[3.5]nonan-2-yl)-2-fluorobenzoic acid (34)

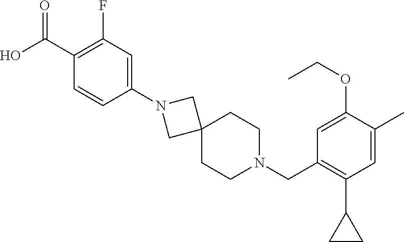 [SMILES: CCOc1cc(CN2CCC3(CC2)CN(c2ccc(C(=O)O)c(F)c2)C3)c(C2CC2)cc1C]

The preparation method was the same as that in Example 19 except that A10a was replaced by 33a. ¹H NMR (500 MHz, DMSO-d6) δ 7.61 (dd, J=8.4, 1.8 Hz, 1H), 7.49 (dd, J=13.5, 1.8 Hz, 1H), 7.06 (s, 1H), 6.83 (s, 1H), 6.52 (t, J=8.8 Hz, 1H), 4.43 (d, J=5.1 Hz, 2H), 4.03 (q, J=7.0 Hz, 2H), 3.96 (s, 2H), 3.79 (s, 2H), 3.35 (d, J=11.9 Hz, 2H), 3.20-3.10 (m, 2H), 2.16 (d, J=13.9 Hz, 2H), 2.12 (s, 3H), 2.07 (tt, J=5.4, 3.0 Hz, 1H), 1.93 (dt, J=13.0, 7.0 Hz, 2H), 1.35 (t, J=7.0 Hz, 3H), 0.97-0.90 (m, 2H), 0.66-0.60 (m, 2H). (ESI, m/z): 453.3[M+H]⁺.

### Pharmacological Experiments

Experimental example 1. Testing SSTR5 antagonistic activity in vitro by Calcium flow detection method Experimental principle: After the receptor binds to the ligand and is activated, it can cause the activation of Gα16 protein, which further activates phospholipase C(PLC) to produce IP3 and DAG. IP3 can bind to the IP3 receptors on the endoplasmic reticulum and mitochondria in the cell, thereby causing the release of intracellular calcium. Therefore, measuring the changes in intracellular calcium can be used in the method to detect the activation state of hSSTR5. Fluo-4/AM is a calcium fluorescent probe indicator used to measure calcium ions. As a non-polar lipid-soluble compound, after it enters the cell, the AM group therein dissociates, and Fluo-4 is released under the action of cellular lipase; because Fluo-4 is a polar molecule and is not easy to pass through the lipid bilayer membrane, it can keep Fluo-4 in the cell fora longtime. Finally, the level of Ga protein activation can be reflected by measuring the intensity of the excited fluorescence. Somatostatin, an agonist of SSTR5, can activate SSTR5 receptors and greatly increase the calcium flow response. The SSTR5 antagonist to be tested can inhibit the agonist activity of the SSTR5 receptor and reduce the calcium increased flow response of the receptor stimulated by the agonist.

### Experimental Steps:

(1) The hSSTR5-CHO-Gα16 and mSSTR5-CHO-Gα16 cell lines stably expressing hSSTR5 or mSSTR5 receptors were seeded at a density of 30,000/well in a 96-well plate and cultured in a 37° C. incubator overnight.
(2) The cells was aspirated off the culture medium, added with freshly prepared dye at 40 μL/well, and incubated in a 37° C. incubator for 45 minutes.
(3) After the dye was aspirated and discarded, the cells were washed with freshly prepared calcium buffer and the culture medium was replaced with 50 μL of calcium buffer.
(4) The SSTR5 agonist Somatostatin-14 was diluted and mixed evenly with calcium buffer.
(5) Detection was performed using a Flextation instrument. Starting from the 15th second, 25 μL of the pre-prepared agonist was automatically added by the instrument, and the fluorescence value at 525 nm was read. The EC₅₀ of the agonist was 9.7 nM, and a concentration of 100 nM agonist was selected for the detection of antagonist activity.
(6) The positive control antagonist and the test compound were diluted and mixed evenly with calcium buffer; wherein the positive control antagonist was 4-(8-((2,6-diethoxy-4′-fluoro-[1,1′-biphenyl]-4-yl)methyl)-3-oxo-2,8-diazaspiro[4.5]dec-2-yl)benzoic acid, which is from the article ACS Med. Chem. Lett. 2018, 9, 1082-1087, corresponding to compound 10.
(7) After the dye was aspirated and discarded, the cells were washed once with freshly prepared calcium buffer, and the culture medium was replaced with 50 μL of the antagonist diluted in a 10-fold concentration gradient.
(8) Detection was performed using a Flextation instrument. Starting from the 15th second, 25 μL of a pre-prepared 100 nM agonist was automatically added by the instrument, and the fluorescence value at 525 nm was read.

### Experimental Results

The in vitro activity test results of the compounds prepared in Examples 1-28 above are shown in Table 1.

TABLE 1

| | hSSTR5 | mSSTR5 |
|---|---|---|
| Example | (IC50) | (IC50) |
| 1 | B | — |
| 2 | A | B |
| 3 | B | — |
| 4 | B | — |
| 5 | B | — |
| 6 | C | — |
| 7 | A | A |
| 8 | B | — |
| 9 | B | — |
| 10 | A | A |
| 11 | B | A |
| 12 | A | A |
| 13 | C | — |
| 14 | A | A |
| 15 | C | — |
| 16 | C | — |
| 17 | B | — |
| 18 | A | — |
| 19 | A | — |
| 20 | B | — |
| 21 | C | — |
| 22 | C | — |
| 23 | C | — |
| 24 | B | — |
| 25 | B | — |
| 26 | C | — |
| 27 | A | A |
| 28 | A | A |
| Positive control | A | A |

Activity ranges are represented by: A: 0.1-50 nM; B: 50-200 nM; C: >200 nM; -: not measured

The above data show that this kind of compound has good SSTR5 antagonistic activity, especially Examples 2, 7, 10, 12, 14, 18, 19, 27 and 28 show potent SSTR5 antagonistic activity.

### Experimental Example 2. Oral Glucose Tolerance (OGTT) Experiment of the Compounds of the Present Invention in C57BL/6 Normal Mice

Experimental method: C57BL/6 mice were fasted for 12 h before the experiment, and the mice were divided into a drug administration group and a blank control group, with 8 mice in each group. 200 μl of distilled water was orally administered to the mice in the blank control group, and 200 μl of an aqueous solution containing the compound of Example 7/positive control drug (same as that in Experimental Example 1) was orally administered to the mice in the drug administration group. Glucose was orally administered (4 g glucose/kg mouse body weight) 60 min after drug administration. Blood was collected from the tail before administration and at 0, 15, 30, 60, 90, and 120 min after administration, and blood glucose was measured using Accu-Chek Advantage II Glucose Monitor (Roche, Indianapolis, IN, USA).

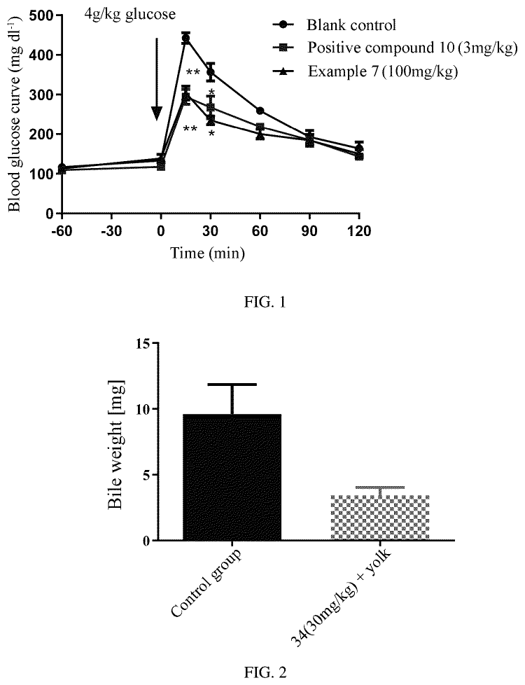

The results of the oral glucose tolerance test (OGTT) test of normal mice after a single dose of the compound are shown in FIG. 1.

Wherein, the positive control is 4-(8-((2,6-diethoxy-4′-fluoro-[1,1′-biphenyl]-4-yl)methyl)-3-oxo-2,8-diazaspiro[4.5]dec-2-yl)benzoic acid, which is from the article ACS Med. Chem. Lett. 2018, 9, 1082-1087.

The above data show that this kind of compounds have a good effect of lowering blood glucose.

### Experimental Example 3. Gallbladder Emptying Promotion Experiment

Experimental method: Before the experiment, 8-10 weeks old C57BL/6J mice were fasted for 17-18 hours and allowed to drink water freely. The mice were then divided into groups according to weight (n=4), and were gavaged with an aqueous solution of the compound prepared in Example 34 (30 mg/kg) or an equal volume of distilled water (control group), with a dosing volume of 10 mL/kg. At one hour after administration, 200 microliters of egg yolk were orally administered to the mice. After 15 minutes, the mice were killed by dislocation and dissected, and the gallbladder was removed and the bile was squeezed out and weighed with an analytical balance.

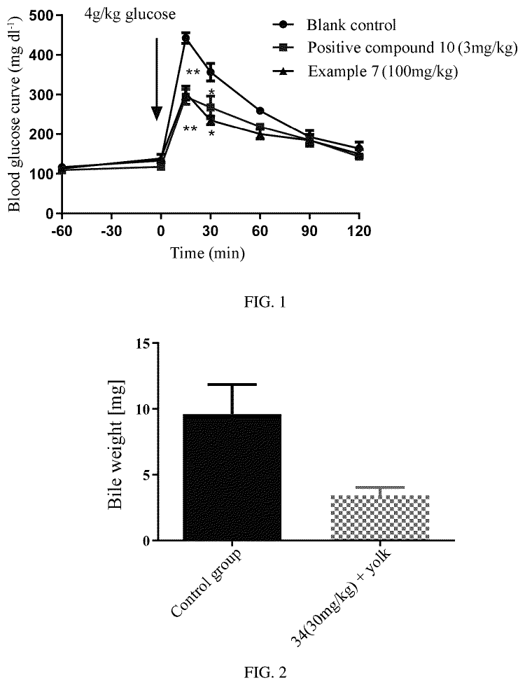

The experimental results are shown in FIG. 2. It can be seen from the FIG. 2 that compared with the control group, the compound of Example 34 of the present disclosure can significantly promote the emptying of the mouse gallbladder. Therefore, it can be used for the prevention and treatment of gallbladder-related diseases such as gallstones, cholestasis, and primary sclerosing cholangitis.

The above is provided only as a specific embodiment of the present invention, but the protection scope of the present invention is not limited thereto. Any person skilled in the art can easily conceive of changes or substitutions within the technical scope disclosed by the present invention, which should be included in the protection scope of the present invention. Therefore, the protection scope of the present invention should be based on the protection scope of the claims.

1. A compound of the following general formula I, or a solvate, hydrate, deuterated compound, stereoisomer, tautomer, or pharmaceutically acceptable salt thereof: 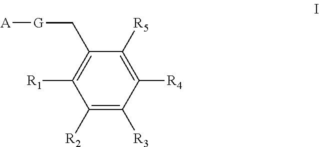
Wherein, R₁, R₂, R₃, R₄, and R₅ are each independently selected from the group consisting of hydrogen, hydroxyl, C₁-C₆ alkyl, C₁-C₆ alkoxy, C₃-C₆ cycloalkyl, halogen, C₁-C₆ haloalkyl, —OH, —NH₂, —N(C₁-C₃ alkyl)(C₁-C₃ alkyl), —NH(C₁-C₃ alkyl), and substituted or unsubstituted C₆-C₁₄ aryl; wherein the substitution means that one or more hydrogen atoms on the aryl group is substituted by a group selected from the group consisting of halogen, C₁-C₃ haloalkoxy, C₁-C₃ alkyl, C₁-C₃ haloalkyl, C₁-C₃ haloalkoxy, C₃-C₆ cycloalkyl, —OH, —NH₂, —NH(C₁-C₃ alkyl), and —N(C₁-C₃ alkyl)(C₁-C₃ alkyl);
alternatively, any two adjacent substituents of R₁, R₂, R₃, R₄ and R₅ together with the benzene ring form a benzo 5-7 membered heterocyclic ring containing N, O or S, or a benzo 5-7 membered carbocyclic ring, wherein the heterocyclic ring or carbocyclic ring is unsubstituted or substituted by one or more groups selected from the group consisting of halogen, C₁-C₆ alkyl, C₁-C₆ haloalkyl, C₁-C₆ alkoxy, C₁-C₆ haloalkoxy, C₃-C₆ cycloalkyl, —OH, —NH₂, and —N(C₁-C₆ alkyl)(C₁-C₆ alkyl);
A-G is represented by the following formula III or IV: 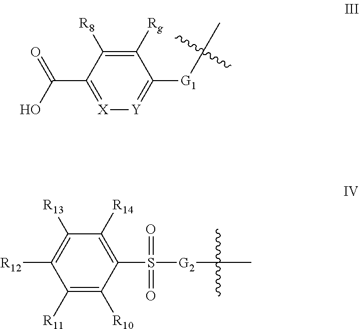
In formula III, R₈ and R₉ are each independently hydrogen, halogen, C₁-C₃ alkyl, C₁-C₃ alkoxy, C₁-C₃ haloalkoxy, —OH, —NH₂, —NH(C₁-C₃ alkyl), or —N(C₁-C₃ alkyl)(C₁-C₃ alkyl);
X and Y are each independently CH or N;
G₁ is selected from the following structures: 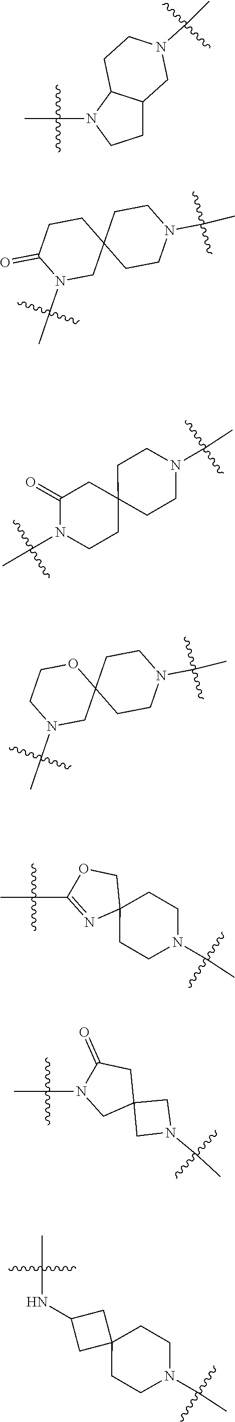 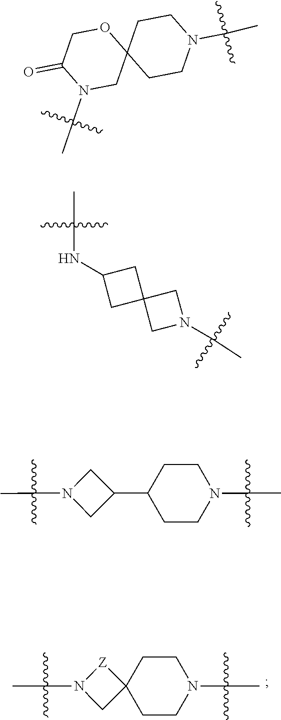
wherein Z is CH₂ or C═O;
In formula IV, R₁₀, R₁₁, R₁₃ and R₁₄ are each independently hydrogen, or halogen; R₁₂ is selected from carboxyl and C₁-C₃ haloalkoxy;
G₂ is selected from the following structures:

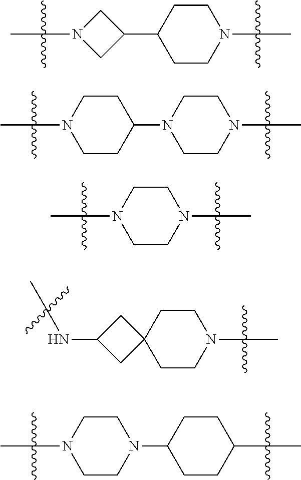

2. The compound, or the solvate, hydrate, deuterated compound, stereoisomer, tautomer, or pharmaceutically acceptable salt thereof according to claim 1,
wherein,
the compound of formula I is represented by the following formula IIIa: 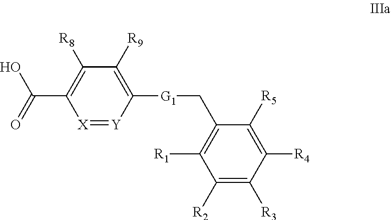
In the formula IIIa,
G₁ is selected from 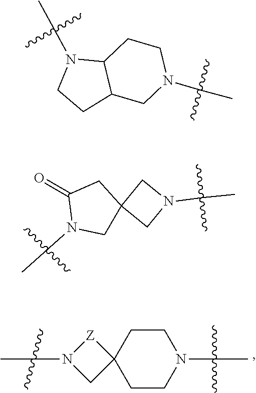
wherein Z is CH₂ or C═O;
R₁ and R₅ are each independently selected from the group consisting of hydrogen, C₃-C₆ cycloalkyl, and substituted or unsubstituted phenyl; wherein the substitution means that the hydrogen on the phenyl is substituted by 1, 2 or 3 groups selected from the group consisting of halogen, C₁-C₃ haloalkoxy, C₁-C₃ haloalkyl, C₁-C₃ alkoxy, and C₁-C₃ alkyl;
R₂ and R₄ are each independently selected from the group consisting of hydrogen, C₁-C₃ alkoxy, halogen, and C₁-C₃ haloalkyl;
R₃ is selected from hydrogen, hydroxyl, halogen, C₁-C₃ alkyl, C₁-C₃ haloalkyl, —N(C₁-C₃ alkyl)(C₁-C₃ alkyl), and substituted or unsubstituted phenyl; wherein the substitution means that the phenyl is substituted by a group selected from the group consisting of halogen, C₁-C₃ haloalkoxy, C₁-C₃ haloalkyl, C₁-C₃ alkoxy, and C₁-C₃ alkyl;
R₈ and R₉ are each independently hydrogen, halogen, C₁-C₃ alkyl, C₁-C₃ alkoxy, or C₁-C₃ haloalkoxy,
X and Y are each independently CH or N.

3. The compound, or the solvate, hydrate, deuterated compound, stereoisomer, tautomer, or pharmaceutically acceptable salt thereof according to claim 1,
wherein,
the compound of formula I may be represented by the following general formula IIIa1: 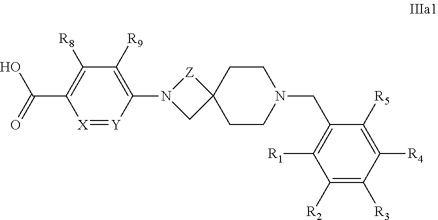
In the general formula IIIa1, each substituent is as defined in claim 1.

4. The compound, or the solvate, hydrate, deuterated compound, stereoisomer, tautomer, or pharmaceutically acceptable salt thereof according to claim 1,
wherein,
the compound of the general formula I is represented by the following general formula IVa: 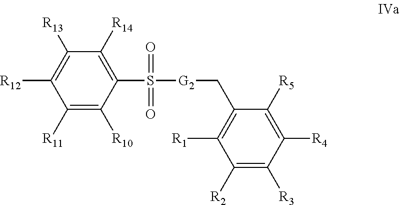
In the general formula IVa,
R₁ and R₅ are each independently selected from the group consisting of hydrogen, C₃-C₆ cycloalkyl, and substituted or unsubstituted phenyl; wherein the substitution means that the hydrogen on the phenyl is substituted by 1, 2 or 3 groups selected from the group consisting of halogen, C₁-C₃ haloalkoxy, C₁-C₃ haloalkyl, C₁-C₃ alkoxy, and C₁-C₃ alkyl;
R₂ and R₄ are each independently selected from the group consisting of hydrogen, C₁-C₃ alkoxy, halogen and C₁-C₃ haloalkyl;
R₃ is selected from hydrogen, halogen, C₁-C₃ alkyl, C₁-C₃ haloalkyl, and substituted or unsubstituted phenyl; wherein the substitution means that the phenyl is substituted by a group selected from the group consisting of halogen, C₁-C₃ haloalkoxy, C₁-C₃ haloalkyl, C₁-C₃ alkoxy, and C₁-C₃ alkyl;
R₁₀, R₁₁, R₁₃ and R₁₄ are each independently hydrogen or halogen,
R₁₂ is selected from carboxyl and C₁-C₃ haloalkoxy, G₂ is as defined in claim 1.

5. The compound, or the solvate, hydrate, deuterated compound, stereoisomer, tautomer, or pharmaceutically acceptable salt thereof according to claim 1, wherein, the compound of the general formula I is selected from one of the following compounds:

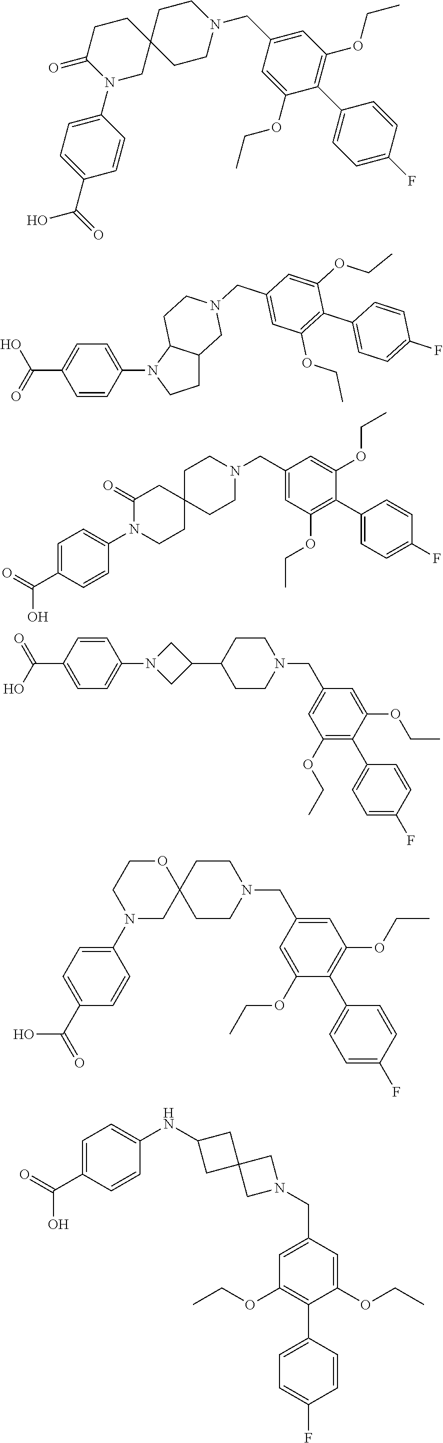

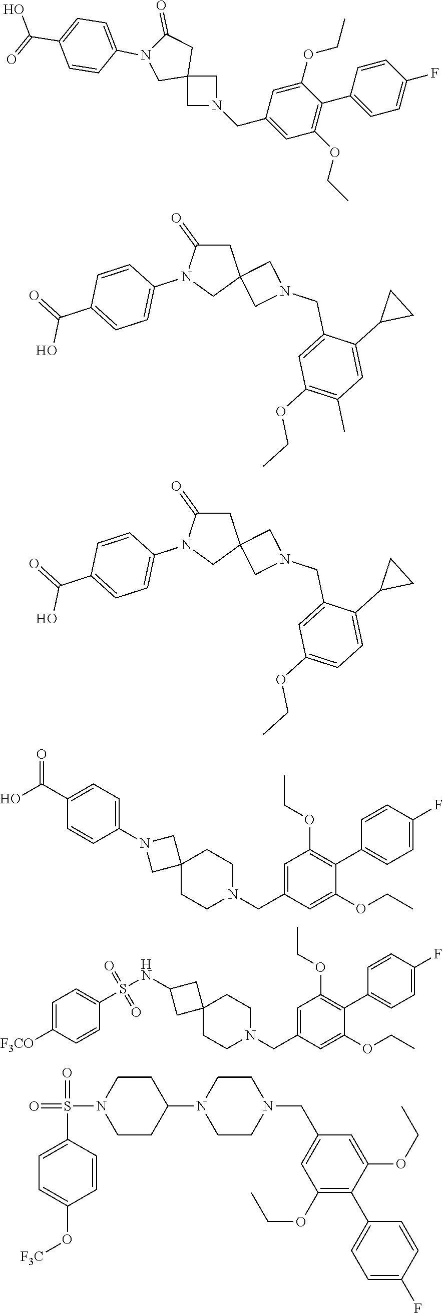

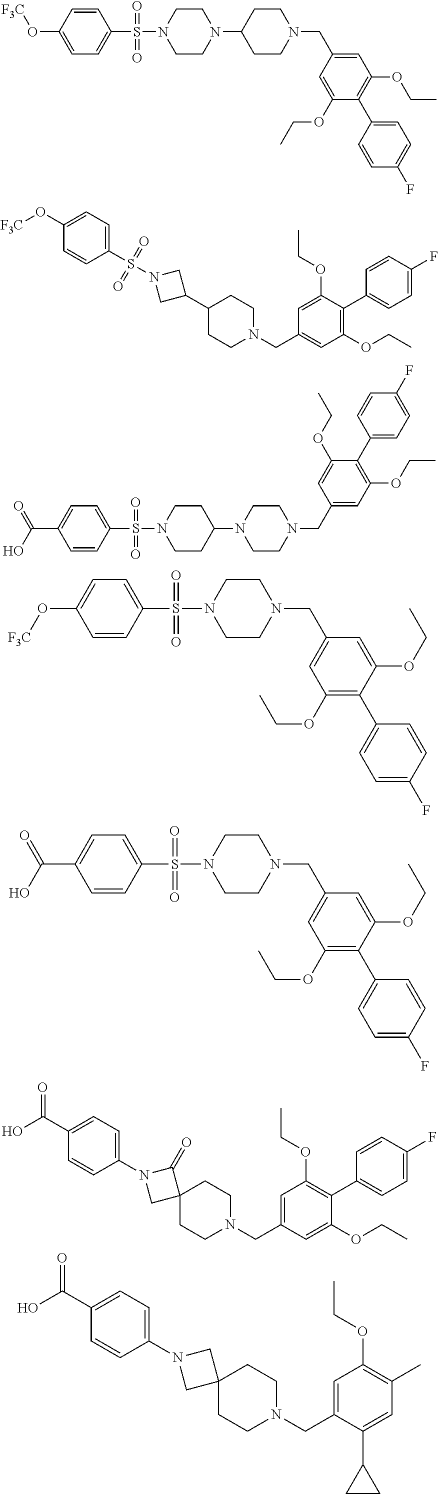

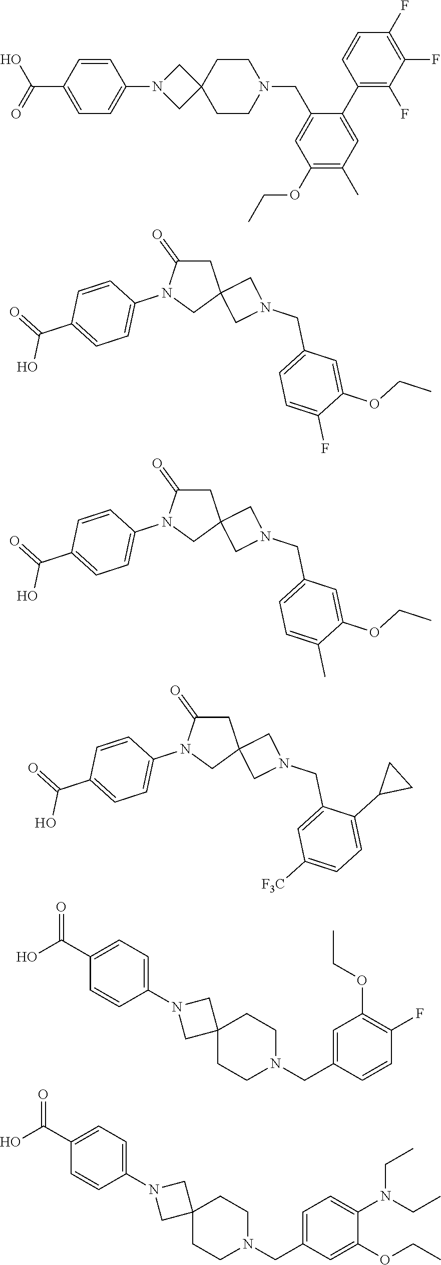

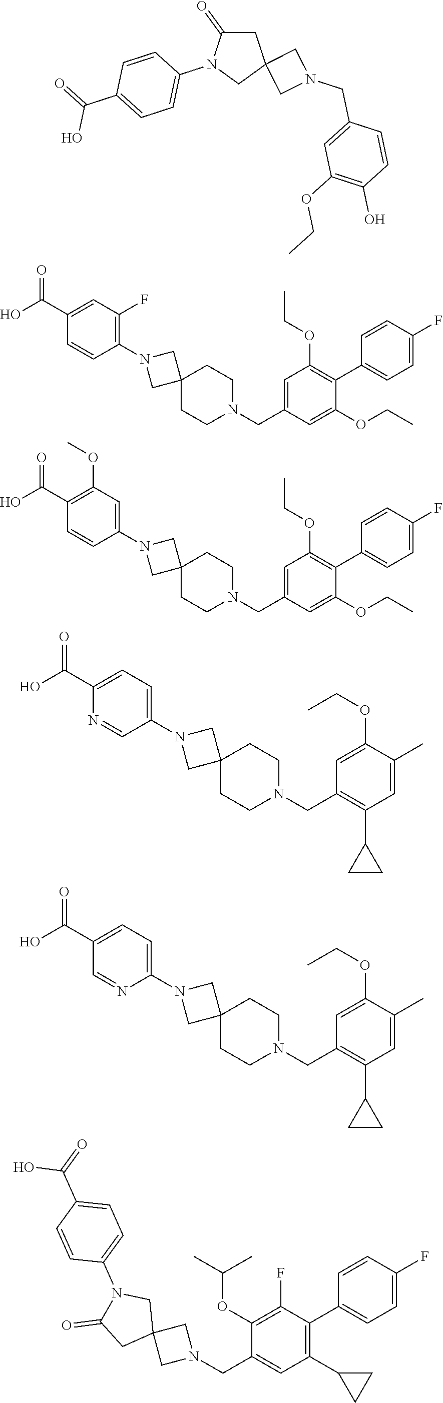

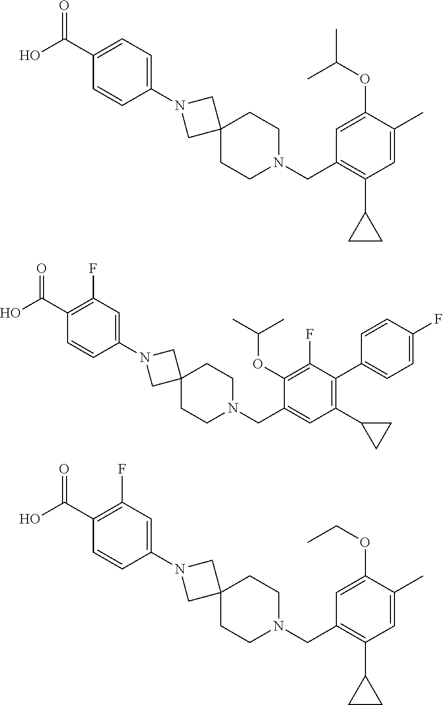

6. A pharmaceutical composition comprising one or more therapeutically effective amounts of the compound of the general formula I or the solvate, hydrate, deuterated compound, stereoisomer, tautomer, pharmaceutically acceptable salt thereof of claim 1, and optionally a pharmaceutically acceptable excipient.

7. The pharmaceutical composition according to claim 6, wherein the pharmaceutical composition further comprises a DPP4 inhibitor and one or more selected from a TGR5 agonist, a GPR40 agonist, a GPR119 agonist, a GPR41 agonist, and a GPR43 agonist.

8. A method of preventing or treating a SSTR5-mediated disease in a subject in need thereof, comprising administering to the subject the compound of claim 1.

9. The method according to claim 8, wherein, the SSTR5-mediated disease comprises type II diabetes, obesity, non-alcoholic fatty liver disease, gallbladder-related diseases, or inflammatory bowel disease.

10. The method according to claim 9, wherein,
the non-alcoholic fatty liver disease is non-alcoholic steatohepatitis;
the gallbladder-related disease is selected from the group consisting of gallstones, primary sclerosing cholangitis, primary biliary cholangitis and cholestasis.

11. The compound, or the solvate, hydrate, deuterated compound, stereoisomer, tautomer, or pharmaceutically acceptable salt thereof according to claim 1, wherein, the compound of formula I is represented by the following formula IIIa: 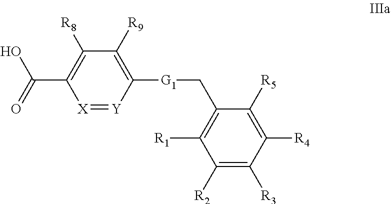
wherein,
G₁ is selected from 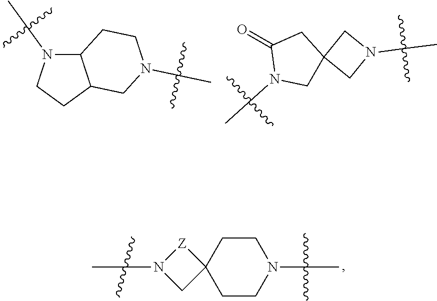
wherein Z is CH₂ or C═O;
R₁ and R₅ are each independently selected from hydrogen, C₃-C₆ cycloalkyl, and phenyl unsubstituted or substituted by 1, 2 or 3 halogens;
R₂ and R₄ are each independently selected from hydrogen, C₁-C₃ alkoxy, and C₁-C₃ haloalkyl;
R₃ is selected from hydrogen, hydroxyl, halogen, C₁-C₃ alkyl, —N(C₁-C₃ alkyl)(C₁-C₃ alkyl), and phenyl unsubstituted or substituted by halogen;
R₈ and R₉ are each independently hydrogen, halogen, or C₁-C₃ alkoxy,
X and Y are each independently CH or N.

12. The compound, or the solvate, hydrate, deuterated compound, stereoisomer, tautomer, or pharmaceutically acceptable salt thereof according to claim 1, wherein, the compound of formula I is represented by the following formula IIIa: 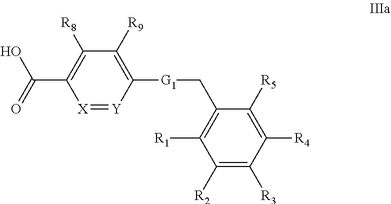
wherein,
G₁ is selected from 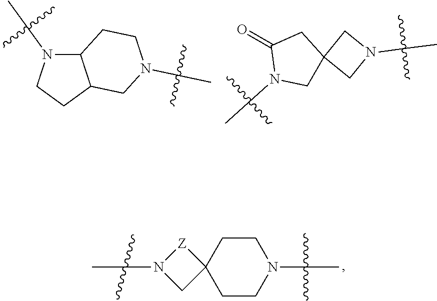
wherein Z is CH₂ or C═O;
R₁ and R₅ are selected from hydrogen, cyclopropyl, and phenyl substituted by 1 to 3 fluorines;
R₂ and R₄ are selected from hydrogen, ethoxy, and trifluoromethyl;
R₃ is selected from hydrogen, hydroxyl, fluorine, methyl, diethylamino, and p-fluorophenyl;
R₈ to R₉ are each independently selected from hydrogen, fluorine, and methoxy,
X and Y are each independently CH or N.

13. The compound, or the solvate, hydrate, deuterated compound, stereoisomer, tautomer, or pharmaceutically acceptable salt thereof according to claim 1, wherein, the compound of formula I is represented by the following formula IIIa: 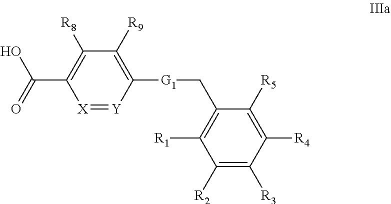
wherein,
G₁ is selected from 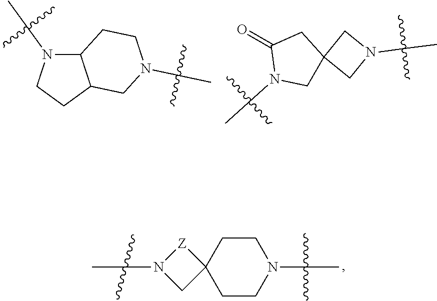
wherein Z is CH₂ or C═O;
R₁ and R₅ are hydrogen; R₂ and R₄ are ethoxy; R₃ is p-fluorophenyl; R₈ and R₉ are each independently selected from hydrogen, fluorine and methoxy, X and Y are each independently CH or N.

14. The compound, or the solvate, hydrate, deuterated compound, stereoisomer, tautomer, or pharmaceutically acceptable salt thereof according to claim 1, wherein, the compound of formula I is represented by the following formula IIIa: 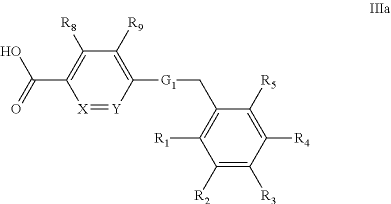
wherein,
G₁ is selected from 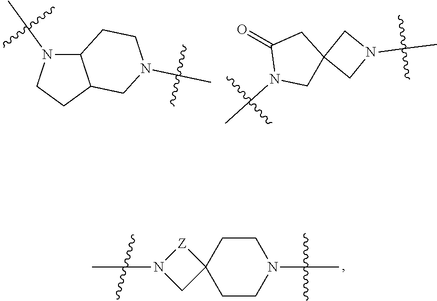
wherein Z is CH₂ or C═O;
one of R₁ and R₅ is hydrogen, and the other is cyclopropyl; one of R₂ and R₄ is ethoxy, and the other is hydrogen; R₃ is methyl; R₈ and R₉ are each independently selected from hydrogen, fluorine, and methoxy, and X and Y are each independently CH or N.

15. The compound, or the solvate, hydrate, deuterated compound, stereoisomer, tautomer, or pharmaceutically acceptable salt thereof according to claim 1, wherein, the compound of formula I may be represented by the following general formula IIIa1: 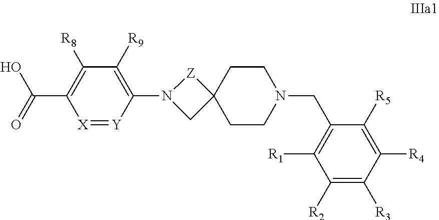
wherein,
R₁, R₂, R₄ and R₅ are each independently hydrogen, halogen, C₁-C₃ alkoxy, C₃-C₆ cycloalkyl, C₁-C₃alkyl, or phenyl substituted by 1 to 3 halogens;
R₃ is hydrogen, C₁-C₃ alkyl, or phenyl substituted by 1 to 3 halogens;
R₈ and R₉ are each independently hydrogen, fluorine, C₁-C₃ alkoxy, or C₁-C₃ alkyl;
Z is CH₂ or C═O;
X and Y are each independently CH or N.

16. The compound, or the solvate, hydrate, deuterated compound, stereoisomer, tautomer, or pharmaceutically acceptable salt thereof according to claim 1, wherein, the compound of the general formula I is represented by the following general formula IVa: 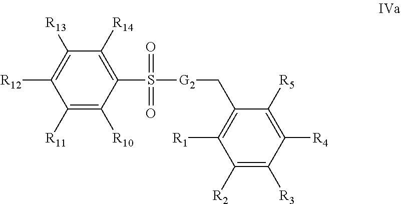
wherein,
R₁ and R₅ are hydrogen; R₂ and R₄ are ethoxy; R₃ is p-fluorophenyl; R₁₂ is selected from C₁-C₃ haloalkoxy and carboxyl; R₁₀, R₁₁, R₁₃ and R₁₄ are all hydrogen.

17. The compound, or the solvate, hydrate, deuterated compound, stereoisomer, tautomer, or pharmaceutically acceptable salt thereof according to claim 1, wherein,
the compound of the general formula I is represented by the following general formula IVa: 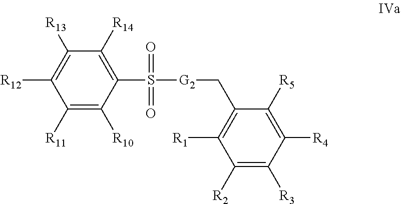
wherein,
R₁ and R₅ are hydrogen; R₂ and R₄ are ethoxy; R₃ is p-fluorophenyl; R₁₂ is selected from C₁-C₃ haloalkoxy and carboxyl; R₁₀, R₁₁, R₁₃ and R₁₄ are all hydrogen; G₂ is selected from

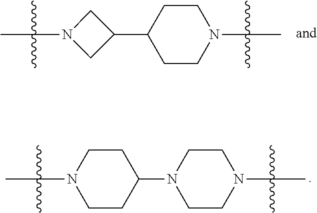 [SMILES: *N1CCC(C2CN(*)C2)CC1 ; *N1CCC(N2CCN(*)CC2)CC1]

18. The compound, or the solvate, hydrate, deuterated compound, stereoisomer, tautomer, or pharmaceutically acceptable salt thereof according to claim 1, wherein, the compound of the general formula I is represented by the following general formula IVa: 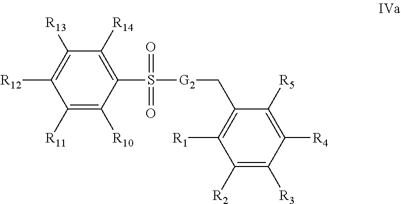
wherein,
R₁ and R₅ are hydrogen; R₂ and R₄ are ethoxy; R₃ is p-fluorophenyl; R₁₂ is trifluoromethoxy or carboxyl, R₁₀, R₁₁, R₁₃ and R₁₄ are all hydrogen.

19. The pharmaceutical composition according to claim 6, wherein the compound of the general formula I is represented by the following general formula IIIa1:

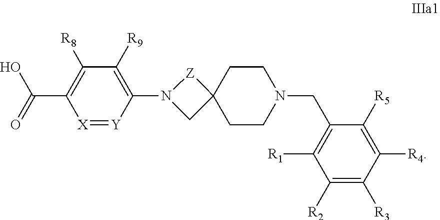

20. The method according to claim 8, wherein, the compound of the general formula I is represented by the following general formula IIIa1: [chemical structure]
